(19) United States
(12) Patent Application Publication
(10) Pub. No.: US 2017/0273968 A1
(43) Pub. Date: Sep. 28, 2017

(54) MAO-B INHIBITORS AND REHABILITATION

(71) Applicant: DART NEUROSCIENCE (CAYMAN) LTD., Grand Cayman (KY)

(72) Inventors: Timothy Tully, Solana Beach, CA (US); Philip Perera, San Diego, CA (US)

(21) Appl. No.: 15/512,320

(22) PCT Filed: Sep. 17, 2015
(86) PCT No.: PCT/US15/50767

§ 371 Date: Mar. 17, 2017

Related U.S. Application Data
(60) Provisional application No. 62/051,728, filed on Sep. 17, 2014.

(51) Int. Cl. A61K 31/47; A61K 31/444; A61K 9/48; A61K 31/435; A61K 31/4725; A61K 9/20; A61K 31/4375; A61K 31/454

(52) U.S. Cl. CPC A61K 31/47; A61K 31/4375; A61K 31/444; A61K 31/454; A61K 31/435; A61K 31/4725; A61K 9/2018; A61K 9/2027; A61K 9/2054; A61K 9/2013; A61K 9/2059; A61K 9/485; A61K 9/4858; A61K 9/4866

(57) ABSTRACT

Methods of rehabilitation of neurological disorders, including neurological deficits associated with neurotraumas, such as stroke and traumatic brain injury, and with muscle disorders, that includes administering to a subject a MAO-B inhibitor.

### CROSS REFERENCE TO RELATED APPLICATIONS

This application claims the benefit of U.S. Ser. No. 62/051,728, filed Sep. 17, 2014, the disclosure of which is incorporated herein by reference in its entirety.

### FIELD OF THE INVENTION

The present invention relates to methods of rehabilitation of neurological disorders, including neurological deficits associated with neurotraumas such as stroke and traumatic brain injury, and the use in such methods of MAO-B inhibitors, including certain naphthyridine and quinolone derivatives, isoxazole derivatives, and pyrazole derivatives.

### BACKGROUND

Monoamine oxidase (MAO) is a flavin-dependent enzyme that oxidatively deaminates monoamine neurotransmitters (such as dopamine, serotonin, epinephrine, tyramine, and phenethylamine) as well as exogenous amines found in some foods and drugs. There are two MAO isoforms in humans, MAO-A and MAO-B, which arise from separate genes. (Bach et. al., Proc. Natl. Acad. Sci. USA 1988, 85, 4934-4938). Both isoforms show a similar preference for dopamine. However, MAO-A preferentially metabolizes serotonin and noradrenaline, while MAO-B preferentially metabolizes phenethylamine and various trace amines.

The two isoforms localize to the outer mitochondrial membrane and show distinct patterns of tissue expression. (e.g., Riederer et al., J. Neural Transm. 1978, 43, 217-226; Saura et al., J. Neural Transm. Suppl. 1990, 32, 49-53; and Saura, et al., Neuroscience 1996, 70, 755-774). In peripheral tissues, MAO-A is primarily found in the liver and gastrointestinal tract, whereas MAO-B appears to be the exclusive isoform expressed in in blood platelets. In the brain, levels of MAO-B are higher than MAO-A, and they can increase with age, with significant increases observed after 50 to 60 years of age. (Fowler et. al., J. Neural Transm. 1980, 49, 1-20).

MAO inhibitors were initially used for treating depression. This use originated in the 1950s but declined by the late 1960s due to growing concerns about potential food and drug interactions. These inhibitors were irreversible and non-selective, raising the possibility of hypertensive crises (the “cheese effect”) arising from the failure of irreversibly inhibited enzyme to metabolize dietary amines—namely tyramine, and of liver toxicity resulting from the interaction of the inhibitors with other drugs not metabolized by MAO-B. (e.g., Benture-Ferrer et al., CNS Drugs 1996, 6, 217-236).

Newer MAO inhibitors, such as rasagiline or selegiline, have been approved as monotherapy or adjunct therapy in treating Parkinson's disease. (Oerthel and Quinn, Baillieres Clin. Neurol. 1997, 6, 89-108). Selegiline has also been approved to treat major depression in the form of a transdermal patch. Although these inhibitors are still irreversible, they show improved selectivity for MAO-B, suggesting improved side effect profiles, but they still suffer from limitations owing to their irreversible binding. (e.g., Youdim, et al., Br. J. Pharmacol. 2001, 132, 500-506; Gerlach et al., Eur. J. Pharmacol. 2001, 5, 97-108; Knudsen and Gerber, Consult. Pharm. 2011, 26, 48-51). Selegiline also becomes non-selective at higher doses, inhibiting both MAO-A and MAO-B. (Jankovic and Poewe, Curr. Opin. Neurol. 2012, 25, 433-447; Keating, et al., CNS Drugs 2012, 1, 781-785).

Stroke is a major cause of disability in Western countries and presents an even greater health and economic burden worldwide due to an aging population. The cost of stroke care in many countries exceeds five percent of the healthcare budget. (Mukherjee et al., Neurosurg. 2011, 76, S85-S90; Quinn et al., J. Rehabil. Med. 2009, 41, 99-111; Brainin et al., Lancet. Neurol. 2007, 6, 553-566; and Palmer et al., Cur. Med. Res. 2005, 21, 19-26). How stroke impacts a patient depends on where in the brain it occurs and how much of the brain it damages. Whereas a small stroke may lead to only minor problems, such as arm or leg weakness, a larger stroke may result in paralysis on one side or the inability to speak.

More than two-thirds of stroke patients do not recover completely from a stroke—even if they receive optimal care during the acute stroke phase. (Hacke et al., Lancet 2004, 363, 768-774). Post-stroke therapy is therefore required to optimize functional recovery in the majority of stroke patients. This requirement underscores the need for improved patient interventions after stroke. The present invention meets these and other needs in the art, partly by disclosing a role for MAO-B inhibitors in enhancing rehabilitation in patients who have suffered a neurotrauma disorder, including stroke.

### BRIEF SUMMARY OF THE INVENTION

The present invention provides methods of treating a neurological disorder, including treating a neurological deficit associated with a neurological disorder, by administering to a subject (e.g., a human) in need thereof an effective amount of an MAO-B inhibitor during rehabilitation from the neurological disorder. The neurological disorders include neurotrauma disorders and muscle disorders.

One particular embodiment provides a method of treating a neurological deficit associated with a neurotrauma disorder during rehabilitation, that includes (a) administering to a subject in need thereof an effective amount of an MAO-B inhibitor, (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone). In certain embodiments, the MAO-B inhibitor is administered chronically, for example, in a post-acute setting after the patient has been medically stabilized.

Neurotrauma disorders can include, for example, stroke, traumatic brain injury (TBI), head trauma, and head injury. In certain embodiments, the subject can be a post-trauma patient, such as a post-acute trauma patient. The rehabilitation can be, for example post-trauma rehabilitation, such as post-acute trauma rehabilitation. In certain embodiments, the treatment including administering a MAO-B inhibitor begins no earlier than: about 2 days after onset of the neurotrauma, about 4 days after onset of the neurotrauma, about 1 week after onset of the neurotrauma, about 1 month after onset of the neurotrauma, about 2 months after onset of the neurotrauma, about 6 months after onset of the neurotrauma, or about 1 year after onset of the neurotrauma. In alternative embodiments, the first initial dosage of MAO-B inhibitor in the treatment of the neurotrauma is in patients who are within about 2 days, about 1 week, about 1 month, about 2 months, about 6 months, about 1 year, or more than about 1 year of onset of the neurotrauma.

Another aspect of the present invention provides a method of treating a neurological deficit associated with a stroke that includes administering to a subject (e.g., a human) in need thereof an effective amount of an MAO-B inhibitor during rehabilitation from the stroke. One particular embodiment provides a method of treating a neurological deficit during rehabilitation of stroke that includes (a) administering to a subject in need thereof an effective amount of an MAO-B inhibitor, (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone). In certain embodiments, the MAO-B inhibitor is administered chronically.

The stroke can be, for example, a hemorrhagic stroke or an ischemic stroke. The subject can be a post-stroke patient, such as a post-acute stroke patient. The rehabilitation can be post-stroke rehabilitation, such as post-acute stroke rehabilitation. In certain embodiments, the treatment begins no earlier than about 2 days after onset of the stroke, or no earlier than about 4 days after onset of the stroke, or no earlier than about 1 week after onset of the stroke, or no earlier than about 1 month after onset of the stroke, or no earlier than about 2 months after onset of the stroke, or no earlier than about 6 months after onset of the stroke, or no earlier than about 1 year after onset of the stroke. In other embodiments, the first initial dosage of MAO-B inhibitor in the treatment of stroke is in patients who are within about 2 days, about 1 week, about 1 month, about 2 months, about 6 months, about 1 year, or more than 1 year of onset of the stroke.

Another aspect of the present invention provides a method of treating a neurological deficit associated with TBI that includes administering to a subject (e.g., a human) in need thereof an effective amount of an MAO-B inhibitor during rehabilitation for the TBI. One particular embodiment provides a method of treating a neurological deficit during rehabilitation of TBI that includes (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone). In certain embodiments, the MAO-B inhibitor is administered chronically.

The subject can be, for example, a post-TBI patient (e.g., a post-acute TBI patient) and the rehabilitation can be post-TBI rehabilitation (e.g., post-acute TBI rehabilitation). The TBI can be a penetrating injury, such as that resulting from an explosion; or a closed head injury, such as that resulting from a blast injury. In certain embodiments, the first initial dosage of MAO-B inhibitor in the treatment of TBI (e.g., post-acute TBI rehabilitation) occurs no earlier than: about 2 days after onset of the TBI, about 4 days after onset of the TBI, about 1 week after onset of the TBI, about 1 month after onset of the TBI, about 2 months after onset of the TBI, about 6 months after onset of the TBI, or about 1 year after onset of the TBI. In other embodiments, the first initial dosage of MAO-B inhibitor in the treatment of TBI is in patients who are within about 2 days, about 1 week, about 1 month, about 2 months, about 6 months, about 1 year, or more than 1 year of onset of the TBI.

In any of the methods described herein, the neurological deficit can be a motor deficit or a cognitive deficit. The cognitive deficit can be a deficit in memory formation, such as a deficit in memory formation, including deficits in long-term memory formation. The rehabilitation can include, for example, one or more of physical therapy, occupational therapy, or cognitive therapy.

Another aspect of the present invention provides a method of treating a neurological deficit associated with a muscle disorder, including a non-stroke muscle disorder, comprising administering to a subject (e.g., a human) in need thereof an effective amount of an MAO-B inhibitor during rehabilitation from the non-stroke muscle disorder. One particular embodiment provides a method of treating a neurological deficit during rehabilitation of a non-stroke muscle disorder that includes (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone). In certain embodiments, the MAO-B inhibitor is administered chronically. The non-stroke motor muscle disorder can be selected from, for example, a muscle weakness, or a muscle paralysis. In some embodiments, the weakness or paralysis is due to Bell's palsy, a viral infection, a demyelinating disease, or multiple sclerosis.

Another aspect of the invention provides a method of facilitating muscle re-education, comprising administering to a subject in need thereof an effective amount of an MAO-B inhibitor during rehabilitation from the neurological disorder. In some embodiments, the methods are directed to facilitating muscle re-education during rehabilitation of a muscle disorder, comprising: (a) administering to a subject in need thereof an effective amount of an MAO-B inhibitor; (b) providing muscle re-education training under conditions sufficient to improve performance of a muscle function whose impairment is associated with the disorder; and (c) repeating steps (a) and (b) one or more times, whereby the amount of training sufficient to improve said performance is reduced compared to that produced by training alone (or whereby the performance is improved compared to that produced by training alone). In some embodiments, the neurological disorder is a neurotrauma, such as stroke and TBI. In other embodiments, the neurological disorder is a muscle disorder. In one aspect, the muscle disorder is a muscle weakness or paralysis. In another aspect, the muscle disorder is a non-stroke muscle disorder, such as a non-stroke muscle disorder or non-stroke muscle paralysis. In one aspect, muscle re-education maintains or increases range of motion.

Another aspect of the invention provides a method of rehabilitating a patient suffering from a neurological deficit associated with a neurotrauma disorder that includes medically stabilizing the patient from the neurotrauma disorder, and, after the patient has been medically stabilized, administering to the patient an effective amount of an MAO-B inhibitor during rehabilitation from the neurotrauma disorder. In another aspect, the invention provides a method of rehabilitating a patient suffering from a neurological deficit associated with a neurotrama disorder that includes administering to the patient an effective amount of an MAO-B inhibitor during rehabilitation, wherein the patient has been medically stabilized from the neurotrama disorder. The patient can be medically stabilized prior to the first administration of the MAO-B inhibitor.

In one embodiment, the neurotrauma disorder is a stroke, and the patient is administered the MAO-B inhibitor during post-acute stroke rehabilitation. Alternatively, the neurotrauma disorder can be, for example, a traumatic brain injury (TBI), and the patient is administered the MAO-B inhibitor during post-acute TBI rehabilitation.

In certain embodiments, the treatment including administering the MAO-B inhibitor to a patient begins within about 1 day after the patient is determined to be medically stable from the neurotrauma (e.g., stroke, TBI), or within about 2 days after the patient is determined to be medically stable from the neurotrauma (e.g., stroke, TBI), or within about 4 days after the patient is determined to be medically stable from the neurotrauma (e.g., stroke, TBI), or within about 7 days after the patient is determined to be medically stable from the neurotrauma (e.g., stroke, TBI), or within about 14 days after the patient is determined to be medically stable from the neurotrauma (e.g., stroke, TBI), or within about 21 days after the patient is determined to be medically stable from the neurotrauma (e.g., stroke, TBI), or within about a month after the patient is determined to be medically stable from the neurotrauma (e.g., stroke, TBI), or within about 3 months after the patient is determined to be medically stable from the neurotrauma (e.g., stroke, TBI), or within about 6 months after the patient is determined to be medically stable from the neurotrauma (e.g., stroke, TBI), or within about 1 year after the patient is determined to be medically stable from the neurotrauma (e.g., stroke, TBI).

In certain embodiments, the MAO-B inhibitor is a reversible inhibitor. The MAO-B inhibitor can also be a selective MAO-B inhibitor. In some embodiments, the MAO-B inhibitor has greater than 10-fold, 50-fold, 100-fold, 200-fold, 500-fold, 1000-fold, 2000-fold, or 4000-fold selectivity for MAO-B over MAO-A.

In some embodiments, the MAO-B inhibitor is a chemical entity of Formula (I):

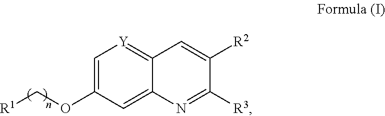

wherein R¹, R², R³, n, and Y have any of the values described herein.

In some embodiments, the MAO-B inhibitor is a chemical entity of Formula (II):

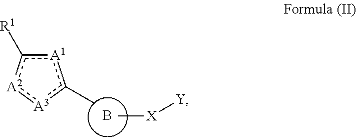

wherein R¹, A¹, A², A³, B, X, and Y have any of the values described herein.

In some embodiments, the MAO-B inhibitor is a chemical entity of Formula (III):

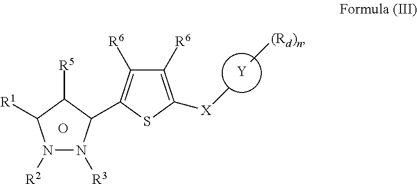

wherein R¹, R², R³, R⁵, R⁶, R^{d}, X, n, and Y have any of the values described herein.

The invention is further directed to the general and specific embodiments defined, respectively, by the claims appended hereto, which are incorporated by reference herein.

### DETAILED DESCRIPTION OF THE INVENTION

The invention can be more fully appreciated by reference to the following description, including the examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

For the sake of brevity, all publications, including patent applications, patents, and other citations mentioned herein, are incorporated by reference in their entirety. Citation of any such publication, however, shall not be construed as an admission that it is prior art to the present invention.

### Terms and Definitions

The use of headings and subheadings, such as “General,” “Chemistry,” or Formulations,” in this section (or any other section of this application) is solely for convenience of reference and not intended to be limiting.

### General

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. Alternatively, with respect to biological systems or processes, the term “about” can mean within an order of magnitude, within 5 fold, or within 2 fold on either side of a value. Numerical quantities given herein are approximate unless stated otherwise, meaning that the term “about” or “approximately” can be inferred when not expressly stated.

To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity for which that could be obtained under the particular stoichiometric conditions. Concentrations that are given as percentages refer to mass ratios, unless indicated differently.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” “criterion,” “known,” and terms of similar meaning should not be construed as limiting the item described to a given time period or to an item available as of a given time, but they should be read to encompass conventional, traditional, normal, or criterion technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

### Chemistry

The term “alkyl” refers to a fully saturated aliphatic hydrocarbon group. The alkyl moiety can be a straight- or branched-chain alkyl group having from 1 to 12 carbon atoms in the chain. Examples of alkyl groups include, but are not limited to, methyl (Me, which also can be structurally depicted by the symbol, “ [symbol] ”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “acyl” as used herein is represented by —C(O)alkyl, wherein an alkyl group is as defined above.

The term “alkyl ester” as used herein is represented by —C(O)Oalkyl, wherein an alkyl group is as defined above.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “haloalkyl” refers to a straight- or branched-chain alkyl group having from 1 to 12 carbon atoms in the chain optionally substituting hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃.and other groups that in light of the ordinary skill in the art and the teachings provided herein, would be considered equivalent to any one of the foregoing examples.

The term “alkoxy” includes a straight chain or branched alkyl group with an oxygen atom linking the alkyl group to the rest of the molecule. Alkoxy includes methoxy, ethoxy, propoxy, isopropoxy, butoxy, t-butoxy, pentoxy and so on. “Aminoalkyl”, “thioalkyl”, and “sulfonylalkyl” are analogous to alkoxy, replacing the terminal oxygen atom of alkoxy with, respectively, NH (or NR), S, and SO₂.

The term “alkoxycarbonyl” used herein refers to the group —C(O)Oalkyl, where alkyl is as defined above, e.g., methoxycarbonyl, ethoxycarbonyl, and the like.

The term “akanoyloxy” refers to a radical of the formula —OC(O)alkyl where alkyl is as defined above, e.g., acetoxy, propanoyloxy, butanoyloxy, and the like.

The term “cyano” refers to the group —CN.

The term “aryl” refers to a monocyclic, or fused or spiro polycyclic, aromatic carbocycle (ring structure having ring atoms that are all carbon), having from 3 to 12 ring atoms per ring. (Carbon atoms in aryl groups are sp2 hybridized.) Illustrative examples of aryl groups include the following moieties:

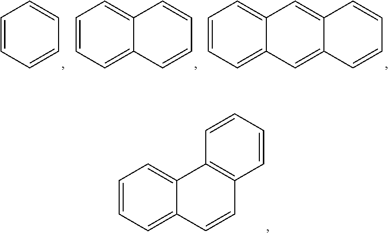

and the like.

The term “cycloalkyl” refers to a saturated or partially saturated carbocycle, such as monocyclic, fused polycyclic, bridged monocyclic, bridged polycyclic, spirocyclic, or spiro polycyclic carbocycle having from 3 to 12 ring atoms per carbocycle. Where the term cycloalkyl is qualified by a specific characterization, such as monocyclic, fused polycyclic, bridged polycyclic, spirocyclic, and spiro polycyclic, then such term cycloalkyl refers only to the carbocycle so characterized. Illustrative examples of cycloalkyl groups include the following entities, in the form of properly bonded moieties:

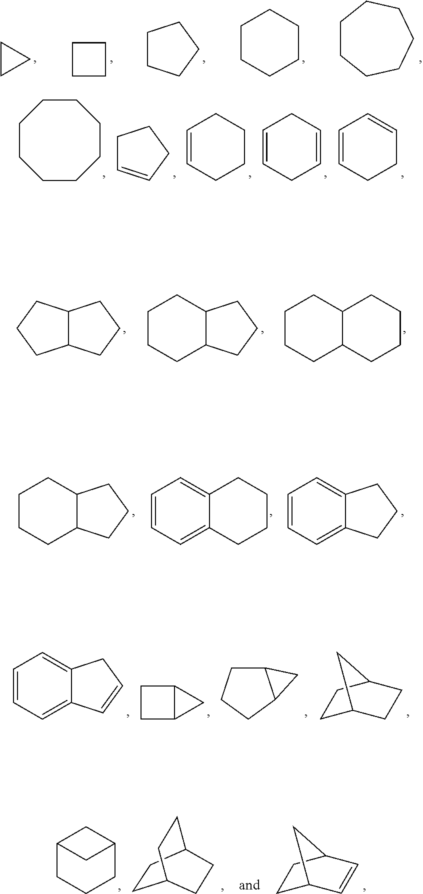

The term “Het” encompasses a radical of a monocyclic, bicyclic, or tricyclic ring system containing a total of 3-20 atoms, including carbon atoms and one or more heteroatoms selected from oxygen, sulfur, and N(X) wherein X is absent or is H, O, (C₁-C₄)alkyl, phenyl or benzyl, wherein one or more ring carbons of Het can optionally be substituted with oxo (═O).

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

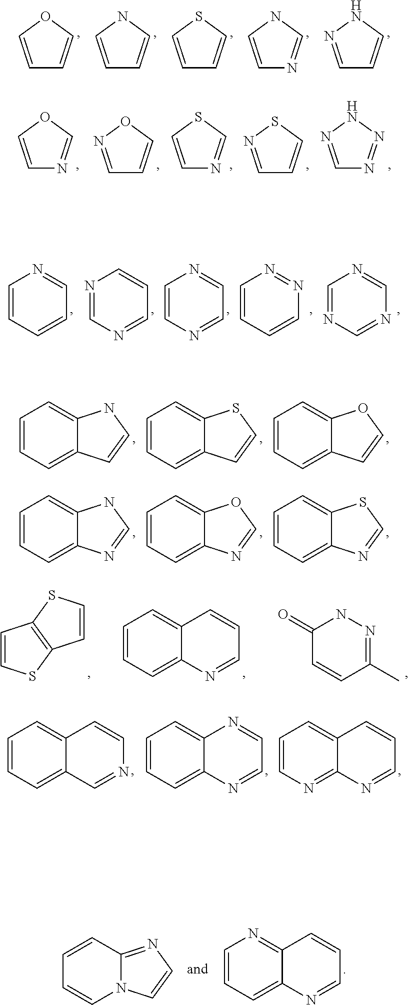

The term, “heterocycle” or “heterocycle group” used herein refers to an optionally substituted monocyclic, bicyclic, or tricyclic ring system, comprising at least one heteroatom in the ring system backbone. The heteroatoms are independently selected from oxygen, sulfur, and nitrogen. The term, “heterocycle” includes multiple fused ring systems. Moreover, the term “heterocycle” includes fused ring systems that may have any degree of saturation provided that at least one ring in the ring system is not aromatic. The monocyclic, bicyclic, or tricyclic ring system may be substituted or unsubstituted, and can be attached to other groups via any available valence, preferably any available carbon or nitrogen. Preferred monocyclic ring systems are of 3 to 8 members. Six membered monocyclic rings contain from up to three heteroatoms wherein each heteroatom is individually selected from oxygen, sulfur, and nitrogen, and wherein when the ring is five membered, preferably it has one or two heteroatoms wherein each heteroatom is individually selected from oxygen, sulfur, and nitrogen. Preferred bicyclic cyclic ring systems are of 7 to 12 members and include spirocycles. An example of an optional substituent includes, but is not limited to, oxo (═O).

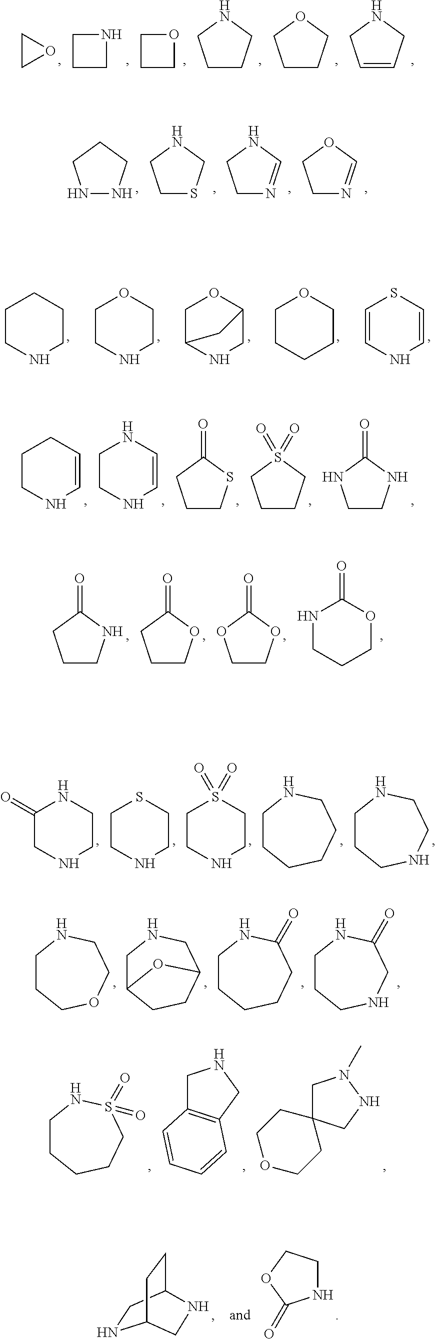

Those skilled in the art will recognize that the exemplary species of aryl, cycloalkyl, heteroaryl, heterocycle and heterocycloalkyl groups listed or illustrated above are not exhaustive, and that additional species within the scope of these defined terms can also be selected.

The term “halogen” represents chlorine, fluorine, bromine or iodine. The term “halo” represents chloro, fluoro, bromo or iodo.

The term “heteroatom” used herein refers to, for example, O (oxygen), S (sulfur), or N (nitrogen).

The terms “para”, “meta”, and “ortho” have meanings as understood in the art. For example, a fully substituted phenyl group has substituents at both “ortho” (o) positions adjacent to the point of attachment of the phenyl ring, both “meta” (m) positions, and the one “para” (p) position across from the point of attachment as illustrated below.

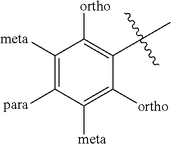

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

### Formulas

Any formula given herein is intended to represent compounds having structures depicted by the structural formulas, as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures can exist as geometric isomers (i.e., cis and trans isomers), as tautomers, or as atropisomers.

As used herein, “tautomer” refers to the migration of a hydrogen or protons between adjacent single and double bonds. The tautomerization process is reversible. Compounds described herein can undergo any possible tautomerization that is within the physical characteristics of the compound. The following is an example tautomerization that can occur in compounds described herein:

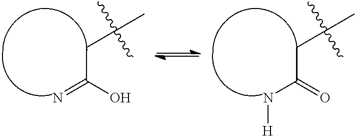 [SMILES: *CC(=N)O ; *CC(N)=O]

The symbols [symbol] and [symbol] are used as meaning the same spatial arrangement in chemical structures shown herein. Analogously, the symbols [symbol] and [symbol] are used as meaning the same spatial arrangement in chemical structures shown herein.

### Chemical Entities

As used herein, the term “chemical entity” collectively refers to a compound, along with the derivatives of the compound, including, but not limited to, salts, chelates, solvates, conformers, crystalline forms/polymorphs, prodrugs, and metabolites (as further defined herein).

As used herein, a “compound” refers to any one of: (a) the actually recited form of such compound; and (b) any of the forms of such compound in the medium in which the compound is being considered when named. For example, reference herein to a compound such as R—COOH encompasses reference to any one of, for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered.

In another example, an expression such as “exposing an entity to a compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form (or forms) that exists in the medium in which such reacting takes place, with (b) the chemically relevant form (or forms) of the compound R—COOH that exists in the medium in which such reacting takes place. In this regard, if such entity is for example in an aqueous environment, it is understood that the compound R—COOH is in the same such medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation and deprotonation. No further examples in this regard are provided herein because these interactions and transformations in a given medium are known by any one of ordinary skill in the art.

In another example, a “zwitterionic” compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As is generally well known, a zwitterion or zwitterionic compound is a neutral compound that has formal unit charges of opposite sign. Sometimes these compounds are referred to by the term “inner salts.” Other sources refer to these compounds as “dipolar ions,” although the latter term is regarded by still other sources as a misnomer. As a specific example, aminoethanoic acid (the amino acid glycine) has the formula H₂NCH₂COOH, and exists in some media (in this case in neutral media) in the form of the zwitterion +H₃NCH₂COO—. Zwitterions, zwitterionic compounds, inner salts, and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

Isotopes can be present in the compounds described. Each chemical element present in a compound either specifically or generically described herein can include any isotope of said element. Any formula given herein is also intended to represent unlabeled forms as well as isotopically-labeled forms of the compounds. Isotopically-labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹⁵N ¹⁸O, ¹⁷O, ³¹P, ³²P, ³⁵S, ¹⁸F, ³⁶Cl, and ¹²⁵I, respectively.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of species for the same variable elsewhere in the formula, unless otherwise stated.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂ and “S²ₑₓₐₘₚₗₑ is one of S₃ and S₄ is accordingly used herein for the sake of brevity but not by way of limitation. The foregoing first example on substituent terminology, which is stated in generic terms, is meant to illustrate the various substituent assignments described herein. The foregoing convention given herein for substituents extends, when applicable, to members such as A¹, A², A³, R¹, R², R³, R⁵, R⁶, R^{d}, n, B, X, and Y, and any other generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂ and S₃, the listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂ and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as A¹, A², A³, R¹, R², R³, R⁵, R⁶, R^{d}, n, B, X, and Y, and any other generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with the total number N of carbon members in the chain that satisfies n≦N≦m, with m>n.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with An attached to the second member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

### Compositions

The term “composition,” as in pharmaceutical composition, is intended to encompass a product comprising the active ingredient(s) and the inert ingredient(s) (e.g., pharmaceutically acceptable excipients) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an active ingredient or disclosed chemical entity (such as a compound of Formula (I), Formula (II), or Formula (III)) and a pharmaceutically acceptable excipient.

The term “carrier” refers to an adjuvant, vehicle, or excipients, with which the compound is administered. In preferred embodiments of this invention, the carrier is a solid carrier. Suitable pharmaceutical carriers include those described in Remington: The Science and Practice of Pharmacy, 21^{st} Ed., Lippincott Williams & Wilkins (2005).

The term “dosage form,” as used herein, is the form in which the dose is to be administered to the subject or patient. The drug is generally administered as part of a formulation that includes nonmedical agents. The dosage form has unique physical and pharmaceutical characteristics. Dosage forms, for example, can be solid, liquid or gaseous. “Dosage forms” can include, for example, a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form is a solid dosage form, and more specifically, comprises a tablet or capsule.

The term “pharmaceutically acceptable,” as used in connection with compositions of the invention, refers to molecular entities and other ingredients of such compositions that are physiologically tolerable and do not typically produce untoward reactions when administered to an animal (e.g., a human). The term “pharmaceutically acceptable” may also mean approved by a regulatory agency of the Federal or a state government or listed in the U.S. Pharmacopeia or other generally recognized pharmacopeia for use in animals (e.g. mammals), and more particularly in humans.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluents to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable pharmaceutical carriers include those described in Remington: The Science and Practice of Pharmacy, 21^{st} Ed., Lippincott Williams & Wilkins (2005).

A “pharmaceutically acceptable salt” is intended to mean a salt of a free acid or base of a disclosed chemical entity, such as a compound represented by Formula (I), Formula (II), or Formula (III), that is non-toxic, biologically tolerable, or otherwise biologically suitable for administration to the subject. See, generally, G. S. Paulekuhn et al., Trends in Active Pharmaceutical Ingredient Salt Selection based on Analysis of the Orange Book Database, J. Med. Chem. 2007, 50, 6665-6672; Berge et al., Pharmaceutical Salts, J. Pharm. Sci. 1977, 66, 1-19; Stahl and Wermuth (eds), Pharmaceutical Salts; Properties, Selection, and Use: 2nd Revised Edition, Wiley-VCS, Zurich, Switzerland (2011). Examples of pharmaceutically acceptable salts are those that are pharmacologically effective and suitable for contact with the tissues of patients without undue toxicity, irritation, or allergic response. A compound of Formula (I), Formula (II), or Formula (III) may possess a sufficiently acidic group, a sufficiently basic group, or both types of functional groups, and accordingly react with a number of inorganic or organic bases, and inorganic and organic acids, to form a pharmaceutically acceptable salt bases, and inorganic and organic acids, to form a pharmaceutically acceptable salt.

As used herein, the term “inert” refer to any inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” refers to a sterile, pharmaceutical product produced under good manufacturing practices (GMP) that is prepared and presented in a manner such that the composition is not likely to cause any untoward or deleterious effects when orally administered to a subject. Unless specified otherwise, all of the compositions disclosed herein are suitable for oral administration.

### Methods and Uses

As used herein, the term “disorder” is used interchangeably with “disease” or “condition”. For example, a neurological disorder also means a neurological disease or a neurological condition.

As used herein, the term “cognitive impairment” is used interchangeably with “cognitive dysfunction” or “cognitive deficit,” all of which are deemed to cover the same therapeutic indications.

As used herein, the term “motor impairment” is used interchangeably with “motor dysfunction” or “motor deficit,” all of which are deemed to cover the same therapeutic indications.

The terms “treat,” “treating,” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

As used in the present disclosure, the term “effective amount” is interchangeable with “therapeutically effective amount” and means an amount or dose of a compound or composition effective in treating the particular disease, condition, or disorder disclosed herein, and thus “treating” includes producing a desired preventative, inhibitory, relieving, or ameliorative effect. In methods of treatment according to the invention, “an effective amount” of at least one compound is administered to a subject (e.g., a mammal). An “effective amount” also means an amount or dose of a compound or composition effective to modulate activity of MAO-B or an associated signaling pathway. The “effective amount” will vary, depending on the compound, the disease (and its severity), the treatment desired, age and weight of the subject, etc.

The terms “individual,” “subject,” and “patient” are used interchangeably herein and can be a vertebrate, in particular, a mammal, more particularly, a primate (including non-human primates and humans) and further includes a laboratory animal in the context of a clinical trial or screening or activity experiment. Thus, as can be readily understood by one of ordinary skill in the art, the compositions and methods of the present invention are particularly suited to administration to any vertebrate, particularly a mammal, and more particularly, a human.

As used herein, a “control animal” or a “normal animal” is an animal that is of the same species as, and otherwise comparable (e.g., similar age, sex) to the animal that is subjected to the treatment whose efficacy is to be ascertained, but does not undergo such treatment.

By “enhance,” “enhancing” or “enhancement” is meant the ability to potentiate, increase, improve or make greater or better, relative to normal, a biochemical or physiological action or effect. For example, enhancing long term memory formation refers to the ability to potentiate or increase long term memory formation in an animal relative to the normal long term memory formation of the animal or controls. As a result, long term memory acquisition is faster or better retained. Enhancing performance of a cognitive task refers to the ability to potentiate or improve performance of a specified cognitive task by an animal relative to the normal performance of the cognitive task by the animal or controls.

As used herein, the term “training protocol,” or “training,” refers to either “cognitive training” or “motor training,” and in specified embodiments, to “muscle re-education.”

As used herein, the term “post-acute” refers to the period that commences after the subject has been medically stabilized from the disorder or condition that inflicts the subject. Accordingly, for example, “post-acute stroke treatment” refers to stroke treatment that commences after the subject has been medically stabilized from the stroke, and “post-acute TBI treatment” refers to TBI treatment that commences after the subject has been medically stabilized from the TBI. Similarly, “post-acute stroke rehabilitation” refers to stroke rehabilitation that commences after the subject has been medically stabilized from the stroke, and “post-acute TBI rehabilitation” refers to TBI rehabilitation that commences after the subject has been medically stabilized from the TBI. Whether a patient has been medically stabilized can be determined according to ordinary of skill in the art, as such determinations are routinely made in a hospital environment, such as by a physician in charge of the patient at the patient's hospital, based on, for example, a determination that the patient's condition, as determined by, for example, vital signs and other data obtained from the patient relating to the patient's underlying condition (e.g., stroke or TBI), is not one characterized by frequent and unpredictable changes.

Reference will now be made to the embodiments of the present invention, examples of which are illustrated by and described in conjunction with the accompanying drawings and examples. While certain embodiments are described herein, it is understood that the described embodiments are not intended to limit the scope of the invention. On the contrary, the present disclosure is intended to cover alternatives, modifications, and equivalents that can be included within the invention as defined by the appended numbered embodiments.

### Chemical Entities

Chemical entities for use in the methods disclosed herein include compounds and derivatives thereof, which can act as inhibitors of monoamine oxidases. Accordingly, chemical entities include an “MAO inhibitor active ingredient,” which is also referred to herein as an “active ingredient,” “MAO inhibitor,” or MAOL.”

The term “selectively inhibiting” as used herein means that an active ingredient (or a composition that has an active ingredient) inhibits the activity of MAO-B to a greater extent than it inhibits the activity of MAO-A. The selectivity index (S1) can be calculated as MAO-A IC₅₀/MAO-B IC₅₀, wherein the MAO-A and MAO-B enzymatic assays are performed according to the fluorometric method described by Matsumoto and colleagues (Matsumoto et. al., Clin. Biochem. 1985, 18, 126-129) with several modifications, as described in U.S. Pat. No. 8,222,243 (Col. 54, 11 20-42).

In specific embodiments of the invention, the active ingredient inhibits the activity of MAO-B 2 times, 5 times, 10 times, 25 times, 50 times, 100 times, 200 times, 500 times, 1000 times, 2000 times, 3000 times, 4000, or 5000 times more than it inhibits the activity of MAO-A. For compositions having more than one active ingredient, the aggregate effect of the active ingredients, as administered in the composition, is considered.

### Compounds

MAO-B inhibitors are known in the art and, for example, include: phenylcoumarine derivatives (ES2343347, Jul. 28, 2010), substituted azole derivatives (International Publication No. WO 2010098600, Sep. 2, 2010), axabenzoxazole derivatives (WO 2010051196, May 6, 2010), benzopyran derivatives (WO 2006102958, Oct. 5, 2006, pyrrolidinylphenyl benzyl ether derivatives (WO 2006097270, Sep. 21, 2006), benzyloxybenzazepine derivatives (WO 2005039591, May 6, 2005), arylpyrrolidinone derivatives (WO 2004026827, Apr. 1, 2004), substituted oxadiazole derivatives (EP504574, Sep. 23, 1992), and N-phenylalkyl substituted aminocarboxanide derivatives (EP0400495, Nov. 3, 1993).

MAO-B inhibitor compounds also include: naphthyridine and quinolone derivatives disclosed in U.S. Published Application No. 2014/0275548, depicted herein by Formula (I); isoxazole derivatives disclosed in U.S. Pat. No. 8,222,243 (depicted herein by Formula (II); and pyrazole derivatives disclosed in U.S. Pat. No. 8,399,487 (depicted herein by Formula (III). Each of these applications is hereby incorporated by reference in its entirety. Unless otherwise specified in this application, definitions and nomenclature set forth therein apply to this disclosure when MAO-B inhibitors are incorporated into the presently disclosed methods and compositions.

### Derivatives

The present invention also includes derivatives of a compound disclosed herein, including compounds of Formula (I), Formula (II), or Formula (III). Derivatives include, but are not limited to, a salt, solvate, conformer, crystalline form/polymorph, prodrug, or metabolite of a compound disclosed herein.

Salts

Accordingly, in one embodiment the invention includes pharmaceutically acceptable salts of the compounds disclosed herein, including those represented by Formula (I), Formula (II), and Formula (III), and methods that include administering such salts in pharmaceutical compositions.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, borate, nitrate, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-1-sulfonates, naphthalene-2-sulfonates, besylate, mesylate and mandelates.

When the active ingredient, such as a compound of Formula (I), Formula (II), or Formula (III), contains a basic nitrogen, the desired pharmaceutically acceptable salt can be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid or glutamic acid, an aromatic acid, such as benzoic acid, 2-acetoxybenzoic acid, naphthoic acid, or cinnamic acid, a sulfonic acid, such as lauryl sulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

When the active ingredient, such as a compound of Formula (I), Formula (II), or Formula (III), is an acid, such as a carboxylic acid or sulfonic acid, the desired pharmaceutically acceptable salt can be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-D-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

Solvates

In other embodiments, the invention provides a solvate of an instantly disclosed active ingredient, such as a compound of Formula (I), Formula (II) or Formula (III), as the MAO-B inhibitor active ingredient, and the use of such solvates in methods that include administering such solvates in pharmaceutical compositions. Certain compounds of the disclosed active ingredients or pharmaceutically acceptable salts of the disclosed active ingredients can be obtained as solvates. In some embodiments, the solvent is water and the solvates are hydrates.

More particularly, solvates include those formed from the interaction or complexes of compounds of the invention with one or more solvents, either in solution or as a solid or crystalline form. Such solvent molecules are those commonly used in the pharmaceutical art, which are known to be innocuous to the recipient, e.g., water, ethanol, ethylene glycol, and the like. Other solvents can be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like. Hydrates include compounds formed by an incorporation of one or more water molecules.

Conformers and Crystalline Forms/Polymorphs

In other embodiments, the invention provides conformer and crystalline form of a disclosed active ingredient, such as a compound of Formula (I), Formula (II) or Formula (III), as MAO-B inhibitor active ingredient, and the use of these derivatives in methods of rehabilitating a patient from a stroke. A conformer is a structure that is a conformational isomer. Conformational isomerism is the phenomenon of molecules with the same structural formula but different conformations (conformers) of atoms about a rotating bond.

Polymorphs have the same chemical formula but a different solid state or crystal structure. In certain embodiments of the invention, active ingredients are in crystalline form. In addition, certain crystalline forms of disclosed active ingredients or pharmaceutically acceptable salts of disclosed active ingredients can be obtained as co-crystals and used as the active ingredient. In still other embodiments, disclosed active ingredients can be obtained in one of several polymorphic forms, as a mixture of crystalline forms, as a polymorphic form, or as an amorphous form, and used as an active ingredient.

Prodrugs

The invention also relates to, as active ingredients, prodrugs of the disclosed compounds (e.g., prodrugs of the compounds of Formula (I), Formula (II) and Formula (III)), and the use of such pharmaceutically acceptable prodrugs in the instantly disclosed methods, such as methods of rehabilitation of a patient from a stroke (e.g., post-acute stroke rehabilitation).

A “prodrug” is a drug precursor that is initially inactive or partially active and upon administration in vivo undergoes chemical conversion by metabolic processes into an active pharmacological agent. Prodrugs are often useful because, in some situations, they can be easier to administer than the parent drug. They can, for instance, be bioavailable by oral administration whereas the parent is not. The prodrug can also have improved solubility in pharmaceutical compositions over the parent drug.

Exemplary prodrugs include compounds having an amino acid residue, or a polypeptide chain of two or more (e.g., two, three or four) amino acid residues, covalently joined through an amide or ester bond to a free amino, hydroxy, or carboxylic acid group of the disclosed formulas. Examples of amino acid residues include the twenty naturally occurring amino acids, commonly designated by three letter symbols, as well as 4-hydroxyproline, hydroxylysine, desmosine, isodemosine, 3-methylhistidine, norvaline, beta-alanine, gamma-aminobutyric acid, citrulline homocysteine, homoserine, ornithine and methionine sulfone.

Additional types of prodrugs can be produced, for instance, by derivatizing free carboxyl groups of structures of the disclosed formulas as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₆alkyl, C₁₋₆cycloalkyl, phenyl, and phenyl(C₁₋₆alkyl) esters. Preferred esters include methyl esters. Prodrugs can also be prepared by derivatizing free hydroxy groups using groups including hemisuccinates, phosphate esters, dimethylaminoacetates, and phosphoryloxymethyloxycarbonyls, following procedures such as those outlined in Fleisher et al., Adv. Drug Delivery Rev. 1996, 19, 115-130.

Carbamate derivatives of hydroxy and amino groups can also yield prodrugs. Carbonate derivatives, sulfonate esters, and sulfate esters of hydroxy groups can also provide prodrugs. Derivatization of hydroxy groups as (acyloxy)methyl and (acyloxy)ethyl ethers, wherein the acyl group can be an alkyl ester, optionally substituted with one or more ether, amine, or carboxylic acid functionalities, or where the acyl group is an amino acid ester as described above, is also useful to yield prodrugs. Prodrugs of this type can be prepared as described in Robinson et al., J. Med. Chem. 1996, 39, 10-18. Free amines can also be derivatized as amides, sulfonamides or phosphonamides. All of these prodrug moieties can incorporate groups including ether, amine, and carboxylic acid functionalities.

Prodrugs can be determined using routine techniques known or available in the art (e.g., Bundgard (ed.), 1985, Design of prodrugs, Elsevier; Krogsgaard-Larsen et al. (eds.), 1991, Design and Application of Prodrugs, Harwood Academic Publishers).

Metabolites

The present invention also relates to, as the active ingredient, a metabolite of a disclosed compound, such as metabolite of a compound of Formula (I), Formula (II) or Formula (III)), as defined herein, and salts thereof. A “metabolite” means a pharmacologically active product of metabolism in the body of a specified compound. The metabolite is in an isolated form outside the body.

Metabolites of a compound can be determined using routine techniques known or available in the art. For example, isolated metabolites can be enzymatically and synthetically produced (e.g., Bertolini et al., J. Med. Chem. 1997, 40, 2011-2016; Shan et al., J. Pharm. Sci. 1997, 86, 765-767; Bagshawe, Drug Dev. Res. 1995, 34, 220-230; and Bodor, Adv Drug Res. 1984, 13, 224-231)

Accordingly, in some embodiments, a chemical entity is any MAO-B inhibitor compound disclosed herein (including a compound of Formula (I), Formula (II), or Formula (III)), a pharmaceutically acceptable salt thereof, a pharmaceutically acceptable solvate thereof, a pharmaceutically acceptable conformer thereof, a pharmaceutically acceptable prodrug thereof, or a pharmaceutically acceptable crystalline form (or polymorph) thereof.

Isotopes

The invention also includes isotopically-labeled compounds, which are identical to the active ingredients disclosed above (e.g., isotopically-labeled compounds of Formula (I), Formula (II) or Formula (III)), but for the fact that one or more atoms are replaced by an atom having an atomic mass or mass number different from the atomic mass or mass number usually found in nature. Examples of isotopes that can be incorporated into compounds of the invention include isotopes of carbon, chlorine, fluorine, hydrogen, iodine, nitrogen, oxygen, phosphorous, sulfur, and technetium, including C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc.

In one aspect, the present invention provides a method of using compositions containing isotopically-labeled active agents and prodrugs of the disclosed active agents, such as in metabolic studies (e.g., with ¹⁴C); reaction kinetic studies (e.g., with ²H or ³H); detection or imaging techniques, such as positron emission tomography (PET) or single-photon emission computed tomography (SPECT), including drug or substrate tissue distribution assays; and radioactive interventions in patients.

Isotopically-labeled compounds and prodrugs of the invention thereof can be prepared by a person of ordinary skill in the art with benefit of his knowledge and this disclosure. An ¹⁸F or ¹¹C labeled compound can be particularly preferred for PET, and an I¹²³ labeled compound can be particularly preferred for SPECT studies. Further substitution with heavier isotopes such as deuterium (i.e., ²H) can afford certain therapeutic advantages resulting from greater metabolic stability, for example increased in vivo half-life or reduced dosage requirements.

It will be appreciated by those skilled in the art that active ingredients of the invention having a chiral center can exist in, and can be isolated, in optically active and racemic forms. Some of these active ingredients can exhibit polymorphism. It is to be understood that the present invention encompasses, in certain embodiments, any racemic, optically-active, polymorphic, stereoisomeric, or regioisomeric form, or mixtures thereof, of a disclosed active ingredient of the invention, which possess the useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase) and how to determine MAO-B inhibiting activity using the standard tests described herein, or using other similar tests which are well known in the art.

### Chemical Entities of Formula (I):

In some embodiments, the MAO-B inhibitor active ingredient (the “MAO-B inhibitor”) is selected from a chemical entity of Formula (I):

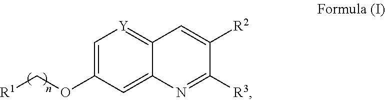

wherein:
n is 1 or 2;
Y is CH or N;
R¹ is a pyridine substituted with —CF₃, or phenyl substituted only in the meta and para positions with a total of one, two, or three R^{a} members;
   each R^{a} is independently selected from the group consisting of halo, —C₁₋₄alkyl, —CF₃, —NO₂, and —OC₁₋₄alkyl;
R² is selected from the group consisting of —C(R^{b})₂R^{c} or —CO—R^{d};
   each R^{b} is independently selected from the group consisting of —H, —F, and —C₁₋₃alkyl, or optionally two R^{b} members are taken together with the carbon to which they are attached to form a C₃₋₆cycloalkyl ring;
   R^{c} is selected from the group consisting of —F, —NH₂, —OH, —OC₁₋₃alkyl, —CH₂OH, —CN, —CO₂—C₁₋₄alkyl, —CO—NHR^{e}, and —C(CH₃)₂OH; provided that when at least one R^{b} is —F then R^{c} is not —F;
   R^{d} is selected from the group consisting of —CH₃, —OC₁₋₄alkyl, —NHR^{e}, and —NHCH₂CH₂N(R^{e})₂,
   each R^{e} is independently —H or —CH₃; and
R³ is selected from the group consisting of —H, —CH₃, —OH, and —CF₃;

In some embodiments, n is 1. In other embodiments, n is 2.

In some embodiments, Y is CH. In other embodiments, Y is N.

In some embodiments, R¹ is 2-(trifluoromethyl)pyridin-4-yl or 6-(trifluoromethyl)pyridin-2-yl. In other embodiments, R¹ is phenyl substituted only in the meta and para positions with a total of, two, or three R^{a} members independently selected from the group consisting of halo, —CF₃, —CH₃, —OCH₃, and —NO₂. In other embodiments, R¹ is selected from the group consisting of 3-chlorophenyl, 3-fluorophenyl, 3-nitrophenyl, 3-methylphenyl, 3-methoxyphenyl, 3-(trifluoromethyl)phenyl, 3-chloro-4-fluorophenyl, 3,4-difluorophenyl, 3-chloro-5-fluorophenyl, 3,5-difluorophenyl, 3-fluoro-5-(trifluoromethyl)phenyl, 3,4,5-trifluorophenyl, 4-chlorophenyl, 4-fluorophenyl, 4-trifluoromethyl)phenyl, 4-fluoro-3-(trifluoromethyl)phenyl, 4-nitrophenyl, 4-methoxyphenyl, 2-(trifluoromethyl)pyridin-4-yl, and 6-(trifluoromethyl)pyridin-2-yl.

In some embodiments, R² is —(CR^{b})₂R^{c}. In other embodiments, R² is —CO—R^{d}. In other embodiments, R² is selected from the group consisting of —CH₂NH₂, —CH₂OH, —CH₂CH₂OH, —CH₂OCH₃, —CH₂CN, —CH₂(C═O)OCH₃, —CH₂(C═O)OCH₂CH₃, —CH₂(C═O)NH₂, —CH₂(CH₃)₂OH, —CH(OH)CH₃, —C(CH₃)₂OH, —C(CH₃)₂CH₂OH, —C(CH₃)₂(C═O)NH₂, —OCH₂CH₃, and —CF(CH₃)₂. In other embodiments, R² is selected from the group consisting of —(C═O)CH₃, —C(═O)OCH₃, —C(═O)OCH₂CH₃, —(C═O)NH₂, —(C═O)NHCH₃, —(C═O)N(CH₃)₂, —C═O)NHCH₂CH₂NH₂, —(C═O)NHCH₂CH₂NHCH₃, and —(C═O)NHCH₂CH₂N(CH₃)₂.

In some embodiments, R^{b} is independently selected from the group consisting of —H, —F and —CH₃. In other embodiments, two R^{b} members are taken together with the carbon to which they are attached to form a cyclopropyl, cyclobutyl, cyclopentyl, or cyclohexyl ring.

In some embodiments, R^{c} is selected from the group consisting of —F, —NH₂, —OH, —OCH₃, —CH₂OH, —CN, —CO₂—C₁₋₄alkyl, —CO—NHR^{e}, and —C(CH₃)₂OH.

In some embodiments, R^{d} is selected from the group consisting of CH₃, —OC₁₋₄alkyl, —NH₂, —NH(CH₃), —NHCH₂CH₂NH(CH₃) and —NHCH₂CH₂N(CH₃)₂.

In some embodiments, R³ is H or —CH₃. In other embodiments, R³ is —CF₃ or —OH.

In some embodiments, the chemical entity is one or more of the group consisting of:

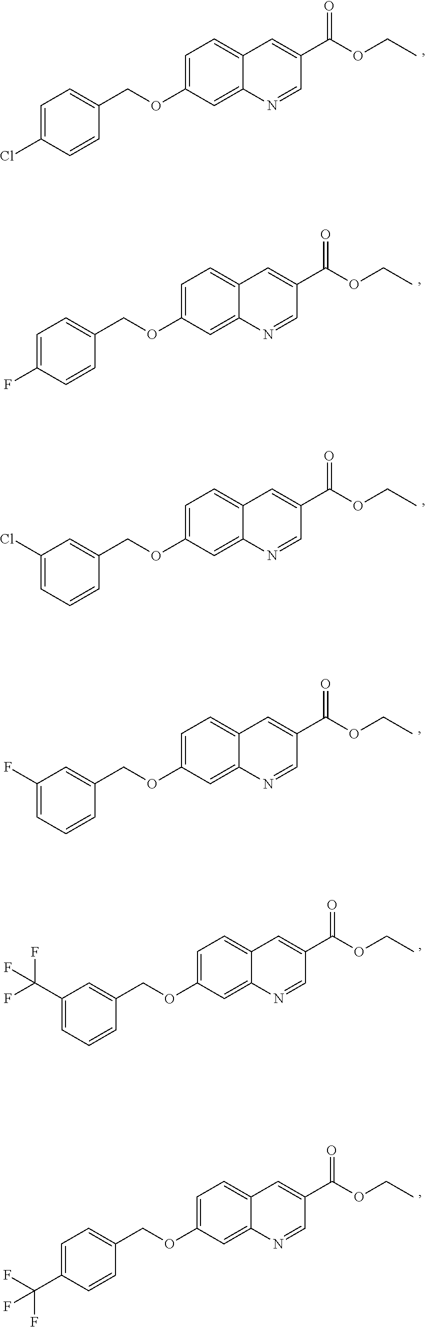 [SMILES: CCOC(=O)c1cnc2cc(OCc3ccc(Cl)cc3)ccc2c1 ; CCOC(=O)c1cnc2cc(OCc3ccc(F)cc3)ccc2c1 ; CCOC(=O)c1cnc2cc(OCc3cccc(Cl)c3)ccc2c1 ; CCOC(=O)c1cnc2cc(OCc3cccc(F)c3)ccc2c1 ; CCOC(=O)c1cnc2cc(OCc3cccc(C(F)(F)F)c3)ccc2c1 ; CCOC(=O)c1cnc2cc(OCc3ccc(C(F)(F)F)cc3)ccc2c1]

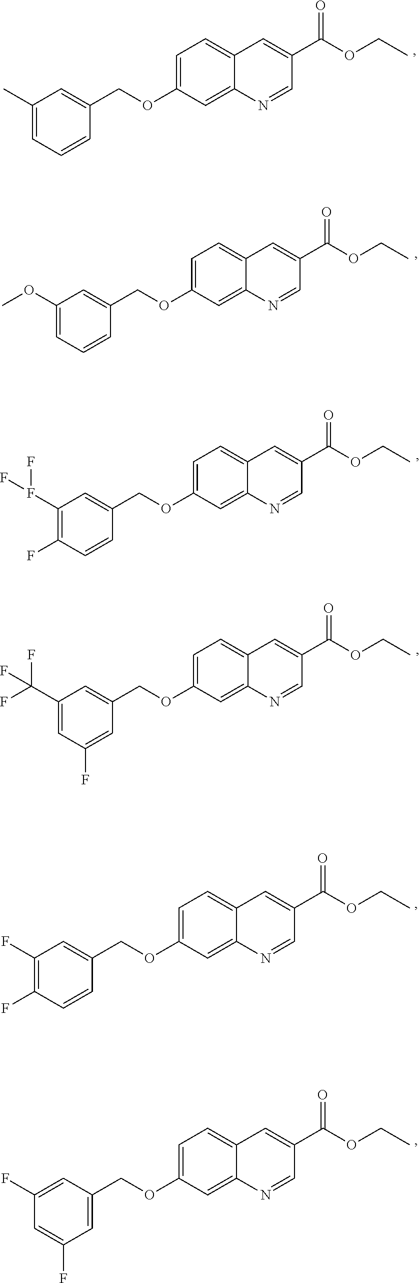

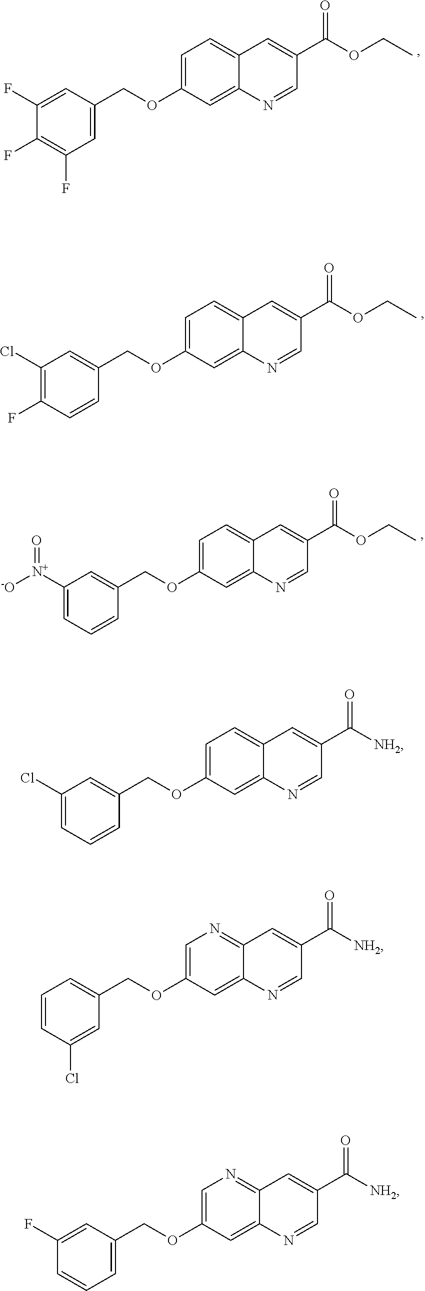

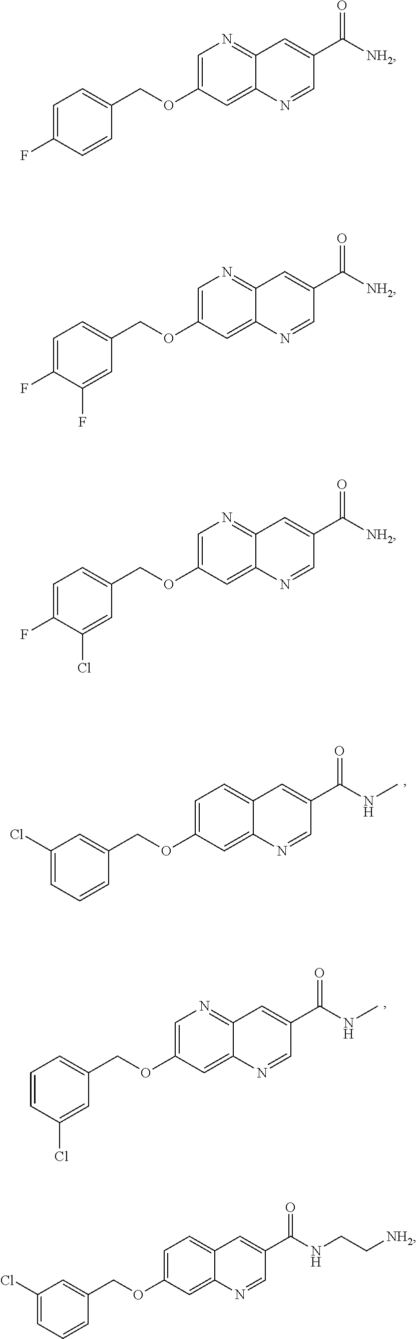 [SMILES: NC(=O)c1cnc2cc(OCc3ccc(F)cc3)cnc2c1 ; NC(=O)c1cnc2cc(OCc3ccc(F)c(F)c3)cnc2c1 ; NC(=O)c1cnc2cc(OCc3ccc(F)c(Cl)c3)cnc2c1 ; CNC(=O)c1cnc2cc(OCc3cccc(Cl)c3)ccc2c1 ; CNC(=O)c1cnc2cc(OCc3cccc(Cl)c3)cnc2c1 ; NCCNC(=O)c1cnc2cc(OCc3cccc(Cl)c3)ccc2c1]

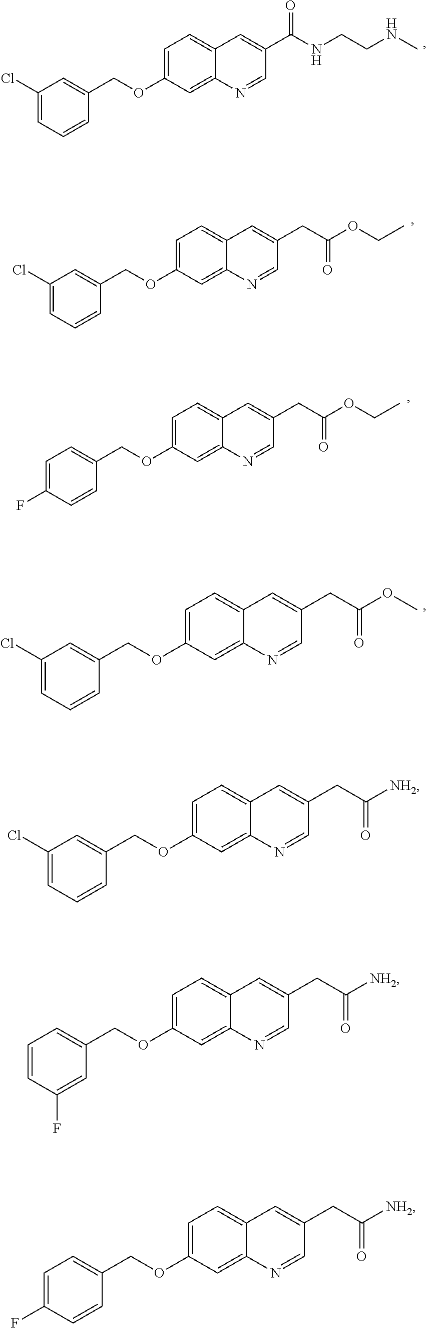

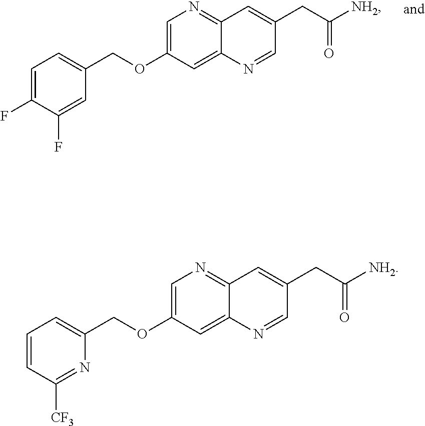 [SMILES: NC(=O)Cc1cnc2cc(OCc3ccc(F)c(F)c3)cnc2c1 ; NC(=O)Cc1cnc2cc(OCc3cccc(C(F)(F)F)n3)cnc2c1]

In some embodiments, the chemical entity can include one or more of the following: a compound of Formula (I), a pharmaceutically acceptable salt of a compound of Formula (I), a pharmaceutically acceptable solvate of a compound of Formula (I), a pharmaceutically acceptable conformer of a compound of Formula (I), a pharmaceutically acceptable prodrug of a compound of Formula (I), and a pharmaceutically acceptable crystalline form (or polymorph) of a compound or Formula (I).

In other embodiments, the chemical entity is a compound of Formula (I), a pharmaceutically acceptable salt of a compound of Formula (I), or a pharmaceutically acceptable prodrug of a compound of Formula (I).

In other embodiments, the chemical entity is a compound of Formula (I) or a pharmaceutically acceptable salt of a compound of Formula (I).

### Chemical Entities of Formula (II):

In some embodiments, the MAO-B inhibitor is selected from a chemical entity of Formula (II):

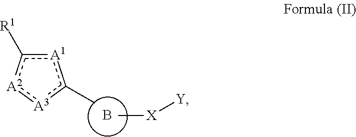

wherein:
R¹ is H (hydrogen), or is selected from the group consisting of aryl and (C₁-C₆)alkyl, each optionally substituted with one or more Rₕ;
   each Rₕ is independently selected from the group consisting of halo, cyano, nitro, and —OH;
A¹ is N (nitrogen), or CR²;
A² and A³ are each independently O (oxygen) or N (nitrogen) with the proviso that when A² is O (oxygen), A³ is N (nitrogen) and when A² is N (nitrogen), A³ is O (oxygen);
   R² is H (hydrogen), (C₁-C₆)alkyl, aryl(C₁-C₆)alkyl, (C₂-C₆)alkenyl, (C₂-C₆)alkynyl, or aryl optionally substituted with one or more halo;
B is aryl or heteroaryl, each optionally substituted with one or more R³;
   each R³ is independently (C₁-C₆)alkyl, or aryl(C₁-C₆)alkyl;
X is —C(═O)—, —C(═S)—, —C(R⁴)₂—, or —S(O)_{z}—;
   each n is independently an integer selected from 0, 1, and 2;
   each z is independently an integer selected from 0, 1, and 2;
Y is R⁴, —N(R⁴)₂, —OR⁴, —SR⁴, or —C(R⁴)₃, each optionally substituted with one or more R_{d};
   each R⁴ is independently selected from the group consisting of hydrogen, —OH, (C₁-C₆)alkyl, (C₂-C₆)alkenyl, (C₂-C₆)alkynyl, (C₁-C₆)alkanoyl, (C₁-C₆)alkoxycarbonyl, (C₃-C₈)cycloalkyl, —(CH₂)ₙ(C₃-C₈)cycloalkyl, heteroaryl, aryl, aryl(C₁-C₆)alkyl, heterocycle, heterocycle(C₁-C₆)alkyl, heterocycle(C₁-C₆)alkanoyl, and NRₐR_{b}; or when Y is —N(R⁴)₂, then two R⁴ groups are optionally taken together with the nitrogen to which they are attached to form a 3-8 membered monocyclic or a 7-12 membered bicyclic ring system, each optionally comprising one or more additional heteroatom groups selected from O (oxygen), S(O)_{z}, and NR_{c} wherein each ring system is optionally substituted with one or more R_{d};
   each Rₐ and R_{b} is independently hydrogen or (C₁-C₆)alkyl, or Rₐ and R_{b} are optionally taken together with the nitrogen to which they are attached to form a 3-8 membered monocyclic or a 7-12 membered bicyclic ring system, each optionally substituted with one or more C₁-C₆alkyl groups;
   each R_{c} is independently selected from the group consisting of hydrogen, (C₁-C₆)alkyl, aryl, heteroaryl, (C₁-C₆)alkylsulfonyl, arylsulfonyl, (C₁-C₆)alkylC(O)—, arylC(O)—, hydroxy(C₁-C₆)alkyl, alkoxy(C₁-C₆)alkyl, heterocycle, (C₁-C₆)alkylOC(O)—, (C₁-C₆)alkylaminocarbonyl, and arylaminocarbonyl;
   each R_{d} is independently halo, cyano, nitro, oxo, R_{f}R_{g}N(C₁-C₆)alkyl, —(CH₂)ₙNR_{f}R_{g}, —C(O)NR_{f}R_{g}, —NRₑC(O)R_{g}, arylC(O)NR_{f}R_{g}, —C(O)OH, (C₁-C₆)alkyl, (C₃-C₈)cycloalkyl, —(CH₂)ₙOH, (C₁-C₆)alkoxy, halo(C₁-C₆)alkoxy, heterocycle, aryl, heterocycle(C₁-C₆)alkyl, aryl(C₁-C₆)alkyl, —NRₑS(O)_{z}(C₁-C₆)alkyl, —NRₑS(O)_{z}aryl, —NRₑC(O)NR_{f}R_{g}, —NRₑC(O)OR_{f}, or —OC(O)NR_{f}R_{g};
   each Rₑ is independently hydrogen, (C₁-C₆)alkyl, aryl or heteroaryl;
   each R_{f} and R_{g} is independently hydrogen, (C₁-C₆)alkyl, aryl or heteroaryl, or R_{f} and R_{g} are optionally taken together with the nitrogen to which they are attached to form a 3-8 membered monocyclic or a 7-12 membered bicyclic ring system, each optionally comprising one or more additional heteroatom groups selected from O (oxygen), S(O)_{z}, and NR_{c} wherein each ring system is optionally substituted with one or more R_{q};
   each R_{q} is independently halo, cyano, nitro, oxo, —NRᵢRⱼ, RᵢRⱼN(C₁-C₆)alkyl, —(CH₂)NRᵢRⱼ, —C(O)NRᵢRⱼ, —NRₖC(O)Rⱼ, arylC(O)NRᵢRⱼ, —C(O)OH, (C₁-C₆)alkyl, (C₃-C₈)cycloalkyl, —(CH₂)ₙOH, (C₁-C₆)alkoxy, halo(C₁-C₆)alkoxy, heterocycle, aryl, heterocycle(C₁-C₆)alkyl, aryl(C₁-C₆)alkyl, —NRₑS(O)_{z}(C₁-C₆)alkyl, —NRₖS(O)_{z}aryl, —NRₖC(O)NRᵢRⱼ, —NRₖC(O) ORᵢ, or —OC(O)NRᵢRⱼ;
   each Rₖ is independently hydrogen, (C₁-C₆)alkyl, aryl or heteroaryl;
   each Rᵢ and Rⱼ is independently hydrogen, (C₁-C₆)alkyl, aryl or heteroaryl; and
      the dashed line represents an optional double bond wherein the ring comprising A¹, A², and A³ is heteroaromatic.

In some embodiments the chemical entity of Formula (II) has the formula:

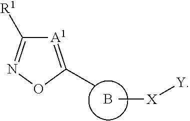

In some embodiments, the chemical entity of Formula (II) has the formula:

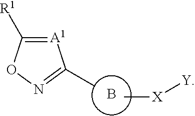

In some embodiments, the chemical entity of Formula (II) has the formula:

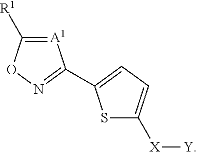

In some embodiments, the chemical entity of Formula (II) has the formula:

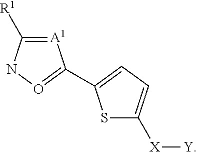

In one embodiment, X is —C(═O). In another embodiment, Y is —N(R⁴)₂; and the two R⁴ groups are taken together with the nitrogen to which they are attached to form a 3-8 membered monocyclic or a 7-12 membered bicyclic ring system, each optionally comprising one or more additional heteroatom groups selected from O (oxygen), S(O)_{z}, and NR_{c} wherein each ring system is optionally substituted with one or more R_{d}.

In some embodiments, the chemical entity has the formula:

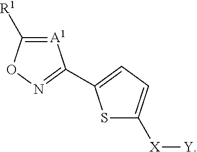

In some embodiments, X is —C(═O). In some embodiments, Y is —N(R⁴)₂; and the two R⁴ groups are taken together with the nitrogen to which they are attached to form a 3-8 membered monocyclic or a 7-12 membered bicyclic ring system, each optionally comprising one or more additional heteroatom groups selected from O (oxygen), S(O)_{z}, and NR_{c} wherein each ring system is optionally substituted with one or more R_{d}.

In some embodiments, the chemical entity is selected from the group consisting of:

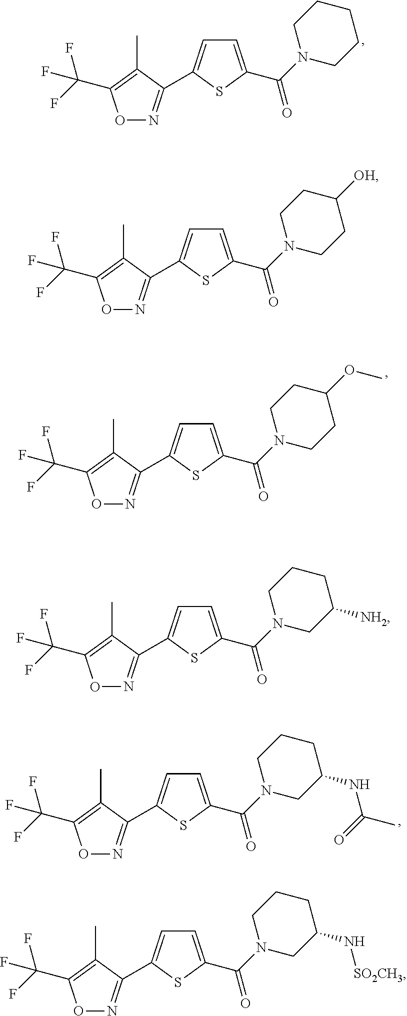 [SMILES: Cc1c(-c2ccc(C(=O)N3CCCCC3)s2)noc1C(F)(F)F ; Cc1c(-c2ccc(C(=O)N3CCC(O)CC3)s2)noc1C(F)(F)F ; COC1CCN(C(=O)c2ccc(-c3noc(C(F)(F)F)c3C)s2)CC1 ; Cc1c(-c2ccc(C(=O)N3CCC[C@H](N)C3)s2)noc1C(F)(F)F ; CC(=O)N[C@H]1CCCN(C(=O)c2ccc(-c3noc(C(F)(F)F)c3C)s2)C1 ; Cc1c(-c2ccc(C(=O)N3CCC[C@H](NS(C)(=O)=O)C3)s2)noc1C(F)(F)F]

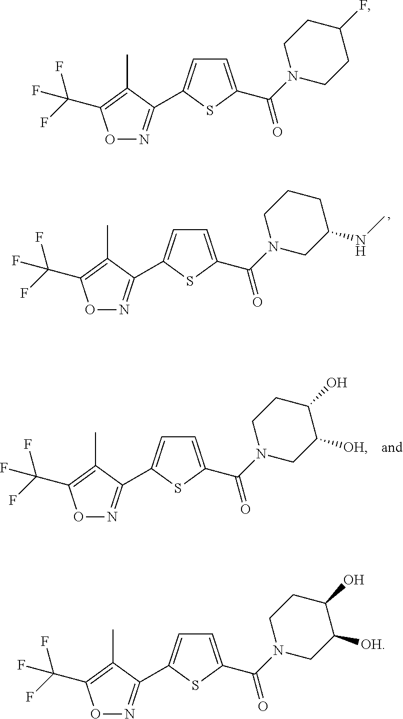 [SMILES: Cc1c(-c2ccc(C(=O)N3CCC(F)CC3)s2)noc1C(F)(F)F ; CN[C@H]1CCCN(C(=O)c2ccc(-c3noc(C(F)(F)F)c3C)s2)C1 ; Cc1c(-c2ccc(C(=O)N3CC[C@H](O)[C@H](O)C3)s2)noc1C(F)(F)F ; "and" ; Cc1c(-c2ccc(C(=O)N3CC[C@@H](O)[C@@H](O)C3)s2)noc1C(F)(F)F]

In some embodiments, the chemical entity is:

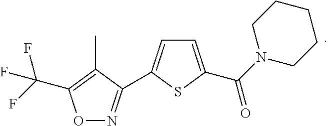 [SMILES: Cc1c(-c2ccc(C(=O)N3CCCCC3)s2)noc1C(F)(F)F]

In some embodiments, the chemical entity is:

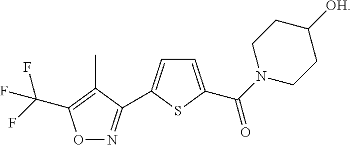 [SMILES: Cc1c(-c2ccc(C(=O)N3CCC(O)CC3)s2)noc1C(F)(F)F]

In some embodiments, the chemical entity is:

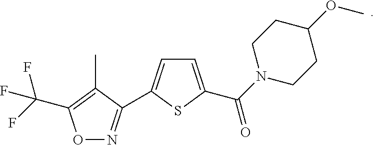 [SMILES: COC1CCN(C(=O)c2ccc(-c3noc(C(F)(F)F)c3C)s2)CC1]

In some embodiments, the chemical entitiy is:

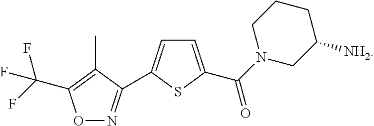 [SMILES: Cc1c(-c2ccc(C(=O)N3CCC[C@H](N)C3)s2)noc1C(F)(F)F]

In some embodiments, the chemical entity is:

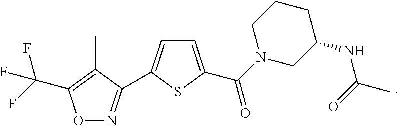 [SMILES: CC(=O)N[C@H]1CCCN(C(=O)c2ccc(-c3noc(C(F)(F)F)c3C)s2)C1]

In some embodiments, the chemical entity is:

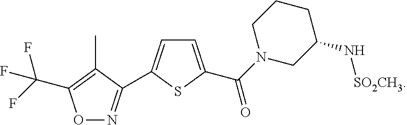 [SMILES: Cc1c(-c2ccc(C(=O)N3CCC[C@H](NS(C)(=O)=O)C3)s2)noc1C(F)(F)F]

In some embodiments, the chemical entity is:

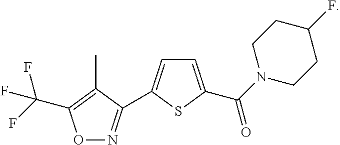 [SMILES: Cc1c(-c2ccc(C(=O)N3CCC(F)CC3)s2)noc1C(F)(F)F]

In some embodiments, the chemical entity is:

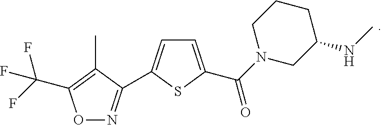 [SMILES: CN[C@H]1CCCN(C(=O)c2ccc(-c3noc(C(F)(F)F)c3C)s2)C1]

In some embodiments, the chemical entity is:

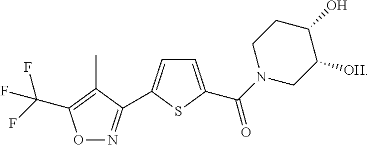 [SMILES: Cc1c(-c2ccc(C(=O)N3CC[C@H](O)[C@H](O)C3)s2)noc1C(F)(F)F]

In some embodiments, the chemical entity is:

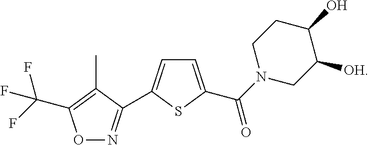 [SMILES: Cc1c(-c2ccc(C(=O)N3CC[C@@H](O)[C@@H](O)C3)s2)noc1C(F)(F)F]

In some embodiments, the chemical entity can include one or more of the following: a compound of Formula (II), a pharmaceutically acceptable salt of a compound of Formula (II), a pharmaceutically acceptable solvate of a compound of Formula (II), a pharmaceutically acceptable conformer of a compound of Formula (II), a pharmaceutically acceptable prodrug of a compound of Formula (II), and a pharmaceutically acceptable crystalline form (or polymorph) of a compound or Formula (II).

In other embodiments, the chemical entity is a compound of Formula (II), a pharmaceutically acceptable salt of a compound of Formula (II), or a pharmaceutically acceptable prodrug of a compound of Formula (II).

In other embodiments, the chemical entity is a compound of Formula (II) or a pharmaceutically acceptable salt of a compound of Formula (II).

### Chemical Entities of Formula (III):

In some embodiments, the MAO-B inhibitor is selected from a chemical entity of Formula (III):

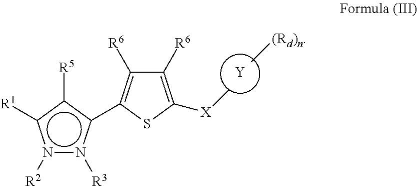

wherein:
R¹ is (C₁-C₆)alkyl, (C₁-C₆)haloalkyl, or phenyl, each of which may be unsubstituted or substituted with one or more R^{e};
one of R² and R³ is absent and the other is hydrogen, (C₁-C₆)alkyl, halo(C₁-C₆)alkyl, hydroxy(C₁-C₆)alkyl, (C₃-C₈)cycloalkyl, amino(C₂-C₆)alkyl, or aryl, each of which may be unsubstituted or substituted with one or more groups selected from alkyl, halo, haloalkyl or nitro, Het, (C₃-C₈)cycloalkyl(C₁-C₆)alkyl, aryl(C₁-C₆)alkyl, or Het(C₁-C₆)alkyl;
X is —C(═O);
Y is piperidine;
n is an integer from 0 to 10 inclusive;
   where each of the n instances of R_{d} is independently halo, hydroxy, cyano, nitro, azido, amino, (C₁-C₆)alkylamino, amino(C₁-C₆)alkyl, amido, (C₁-C₆)alkylamido, aryl amido, carboxylic acid, (C₁-C₆)alkyl, hydroxy(C₁-C₆)alkyl, halo(C₁-C₆)alkyl, (C₁-C₆)alkoxy, halo(C₁-C₆)alkoxy, (C₁-C₆)alkanoyl, (C₁-C₆)alkoxycarbonyl, carboxy, (C₁-C₆)alkanoyloxy, halo(C₁-C₆)alkenyl, Het, aryl, Het(C₁-C₆)alkyl, or aryl(C₁-C₆)alkyl, (C₁-C₆)alkylaryl, sulfonyl, sulfonamido, urea, carbamate, unsubstituted or substituted with one or more substituents R^{e};
   or two members R_{d} come together with the atom to which they are attached to form a ketone or spirocyclic carbocyclic or heterocyclic ring;
   or two R_{d} come together with the atoms to which they are attached to form a bicyclic carbocyclic or heterocyclic ring, wherein each spirocyclic or bicyclic ring is unsubstituted or substituted with one or more halo, hydroxy, cyano, nitro, azido, (C₁-C₆)alkyl, hydroxy(C₁-C₆)alkyl, halo(C₁-C₆)alkyl, (C₁-C₆)alkoxy, halo(C₁-C₆)alkoxy, (C₁-C₆)alkanoyl, (C₁-C₆)alkoxycarbonyl, carboxy, (C₁-C₆)alkanoyloxy, NR_{f}R_{g}, R_{f}R_{g}NC(═O)—, phenyl, or phenyl(C₁-C₆)alkyl, sulfonyl, sulfonamido, urea, carbamate, wherein R_{f} and R_{g} together with the nitrogen to which they are attached form a piperidino, pyrrolidino, morpholino, or thiomorpholino ring, unsubstituted or substituted with one or more substituents R^{e};
   where each Rₑ is independently selected from halo, hydroxy, cyano, nitro, azido, (C₁-C₆)alkyl, Het, aryl, (C₁-C₆)alkylHet, (C₁-C₆)alkylaryl, (C₁-C₆)alkylHet(C₁-C₆)alkyl, (C₁-C₆)alkylaryl(C₁-C₆)alkyl, (C₁-C₆)haloalkyl, (C₁-C₆)alkoxy, (C₁-C₆)haloalkoxy, (C₁-C₆)alkanoyl, (C₁-C₆)alkoxycarbonyl, carboxy, and (C₁-C₆)alkanoyloxy;
R⁵ is H, (C₁-C₆)alkyl, (C₁-C₆)alkenyl, (C₁-C₆)alkynyl, or aryl(C₁-C₆)alkyl; and where each R⁶ is independently selected from H, (C₁-C₆)alkyl, amino, amido, acyl, and aryl(C₁-C₆)alkyl;
wherein the chemical entity is selected from the group consisting of compounds of Formula (III), pharmaceutically acceptable salts of compounds of Formula (III), and pharmaceutically acceptable prodrugs of compounds of Formula (III).

In some embodiments, the chemical entity is selected from the group consisting of:

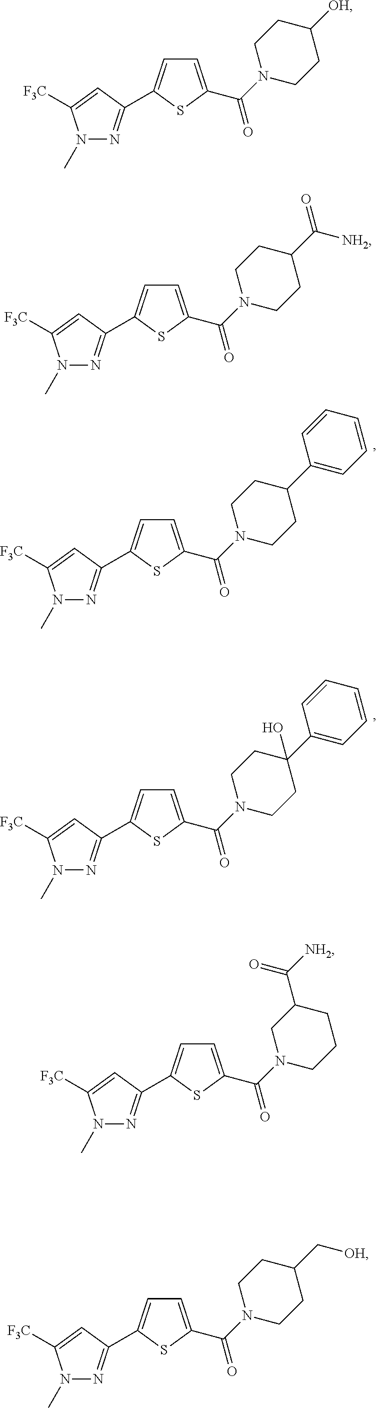 [SMILES: Cn1nc(-c2ccc(C(=O)N3CCC(O)CC3)s2)cc1C(F)(F)F ; Cn1nc(-c2ccc(C(=O)N3CCC(C(N)=O)CC3)s2)cc1C(F)(F)F ; Cn1nc(-c2ccc(C(=O)N3CCC(c4ccccc4)CC3)s2)cc1C(F)(F)F ; Cn1nc(-c2ccc(C(=O)N3CCC(O)(c4ccccc4)CC3)s2)cc1C(F)(F)F ; Cn1nc(-c2ccc(C(=O)N3CCCC(C(N)=O)C3)s2)cc1C(F)(F)F ; Cn1nc(-c2ccc(C(=O)N3CCC(CO)CC3)s2)cc1C(F)(F)F]

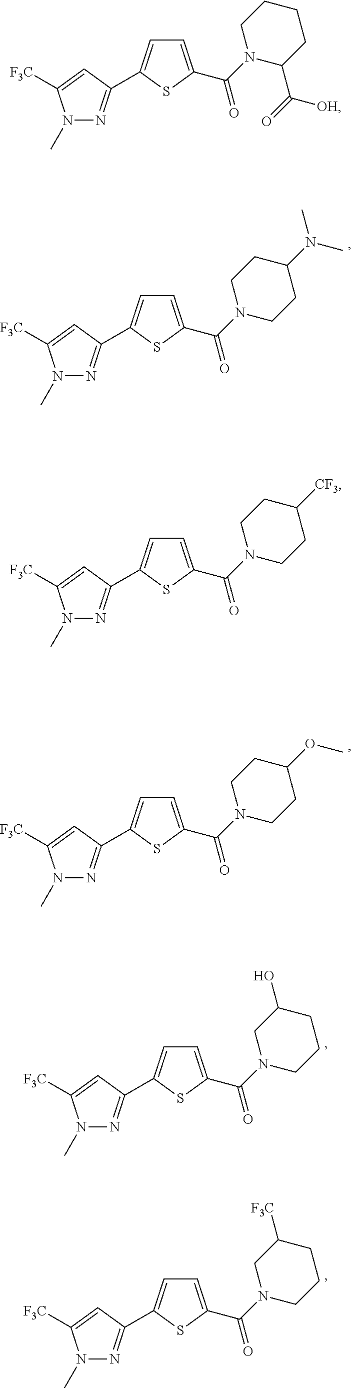 [SMILES: Cn1nc(-c2ccc(C(=O)N3CCCCC3C(=O)O)s2)cc1C(F)(F)F ; CN(C)C1CCN(C(=O)c2ccc(-c3cc(C(F)(F)F)n(C)n3)s2)CC1 ; Cn1nc(-c2ccc(C(=O)N3CCC(C(F)(F)F)CC3)s2)cc1C(F)(F)F ; COC1CCN(C(=O)c2ccc(-c3cc(C(F)(F)F)n(C)n3)s2)CC1 ; Cn1nc(-c2ccc(C(=O)N3CCCC(O)C3)s2)cc1C(F)(F)F ; Cn1nc(-c2ccc(C(=O)N3CCCC(C(F)(F)F)C3)s2)cc1C(F)(F)F]

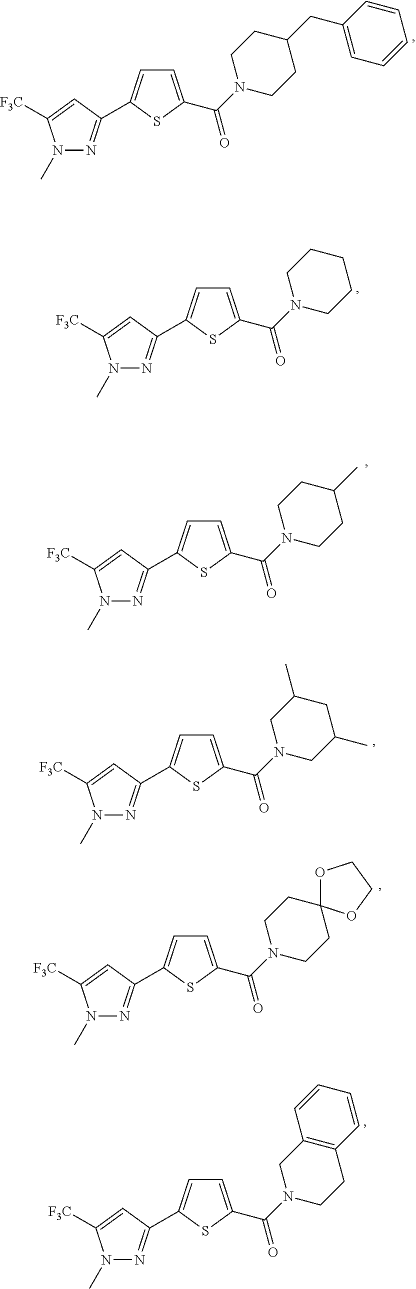 [SMILES: Cn1nc(-c2ccc(C(=O)N3CCC(Cc4ccccc4)CC3)s2)cc1C(F)(F)F ; Cn1nc(-c2ccc(C(=O)N3CCCCC3)s2)cc1C(F)(F)F ; CC1CCN(C(=O)c2ccc(-c3cc(C(F)(F)F)n(C)n3)s2)CC1 ; CC1CC(C)CN(C(=O)c2ccc(-c3cc(C(F)(F)F)n(C)n3)s2)C1 ; Cn1nc(-c2ccc(C(=O)N3CCC4(CC3)OCCO4)s2)cc1C(F)(F)F ; Cn1nc(-c2ccc(C(=O)N3CCc4ccccc4C3)s2)cc1C(F)(F)F]

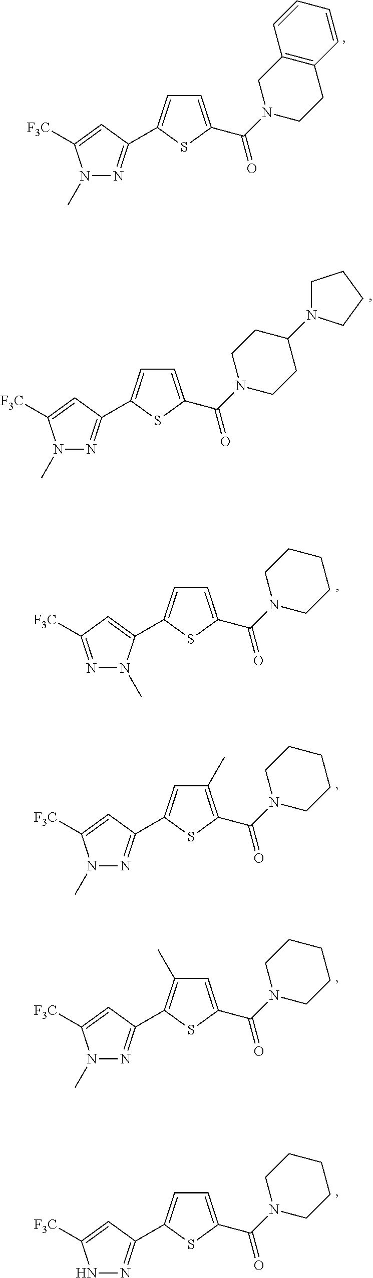 [SMILES: Cn1nc(-c2ccc(C(=O)N3CCc4ccccc4C3)s2)cc1C(F)(F)F ; Cn1nc(-c2ccc(C(=O)N3CCC(N4CCCC4)CC3)s2)cc1C(F)(F)F ; Cn1nc(C(F)(F)F)cc1-c1ccc(C(=O)N2CCCCC2)s1 ; Cc1cc(-c2cc(C(F)(F)F)n(C)n2)sc1C(=O)N1CCCCC1 ; Cc1cc(C(=O)N2CCCCC2)sc1-c1cc(C(F)(F)F)n(C)n1 ; O=C(c1ccc(-c2cc(C(F)(F)F)[nH]n2)s1)N1CCCCC1]

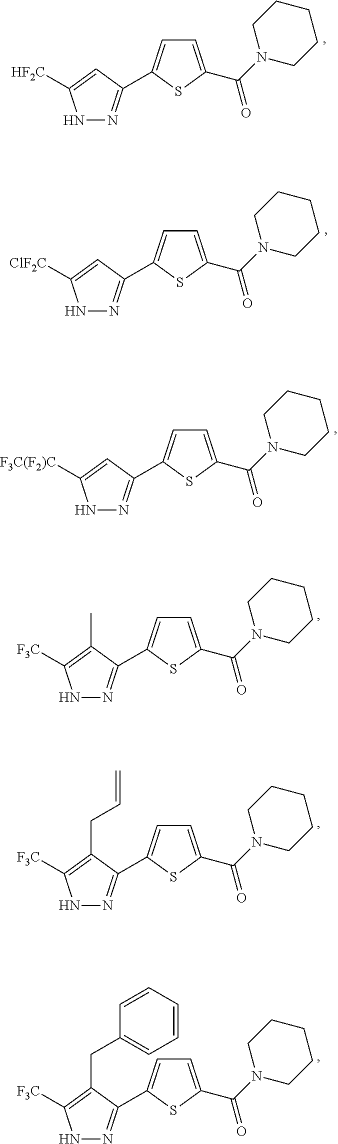

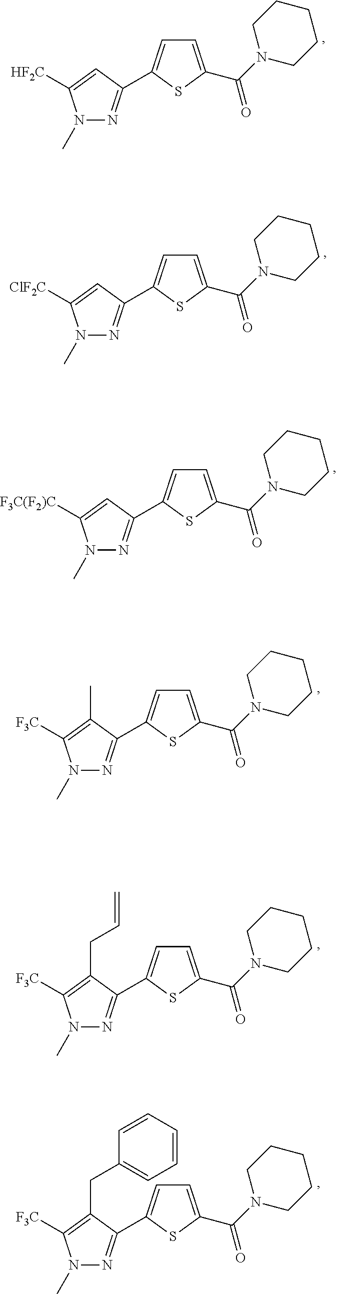

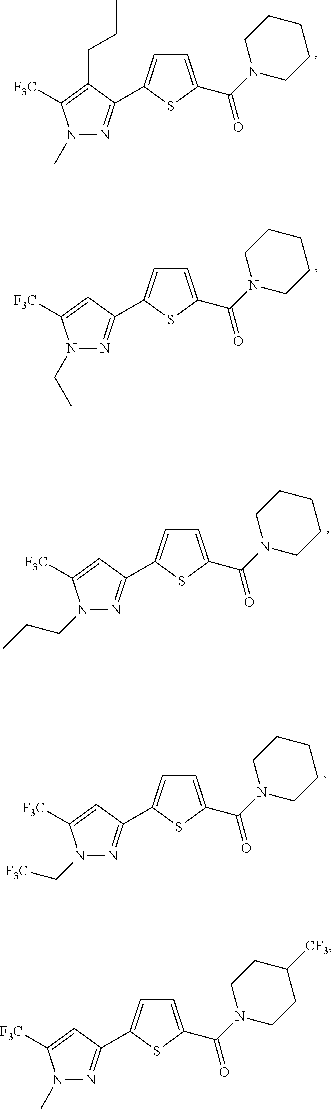 [SMILES: CCCc1c(-c2ccc(C(=O)N3CCCCC3)s2)nn(C)c1C(F)(F)F ; CCn1nc(-c2ccc(C(=O)N3CCCCC3)s2)cc1C(F)(F)F ; CCCn1nc(-c2ccc(C(=O)N3CCCCC3)s2)cc1C(F)(F)F ; O=C(c1ccc(-c2cc(C(F)(F)F)n(CC(F)(F)F)n2)s1)N1CCCCC1 ; Cn1nc(-c2ccc(C(=O)N3CCC(C(F)(F)F)CC3)s2)cc1C(F)(F)F]

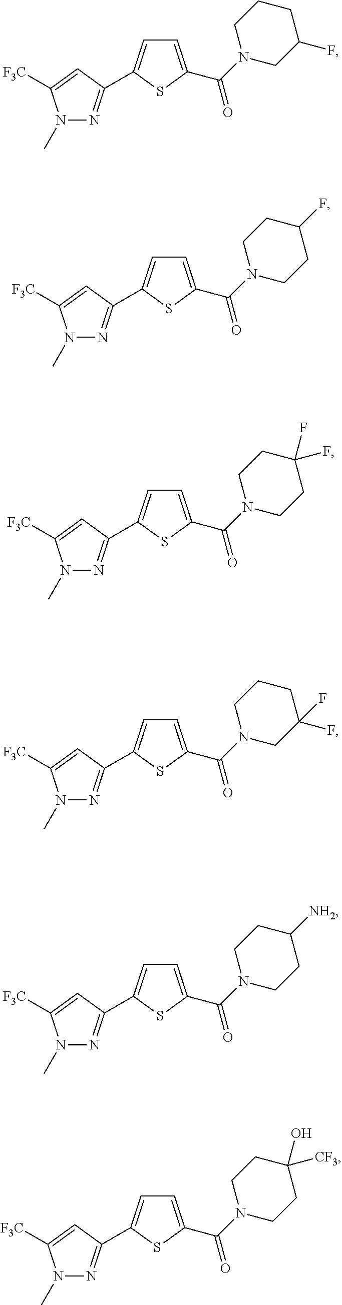 [SMILES: Cn1nc(-c2ccc(C(=O)N3CCCC(F)C3)s2)cc1C(F)(F)F ; Cn1nc(-c2ccc(C(=O)N3CCC(F)CC3)s2)cc1C(F)(F)F ; Cn1nc(-c2ccc(C(=O)N3CCC(F)(F)CC3)s2)cc1C(F)(F)F ; Cn1nc(-c2ccc(C(=O)N3CCCC(F)(F)C3)s2)cc1C(F)(F)F ; Cn1nc(-c2ccc(C(=O)N3CCC(N)CC3)s2)cc1C(F)(F)F ; Cn1nc(-c2ccc(C(=O)N3CCC(O)(C(F)(F)F)CC3)s2)cc1C(F)(F)F]

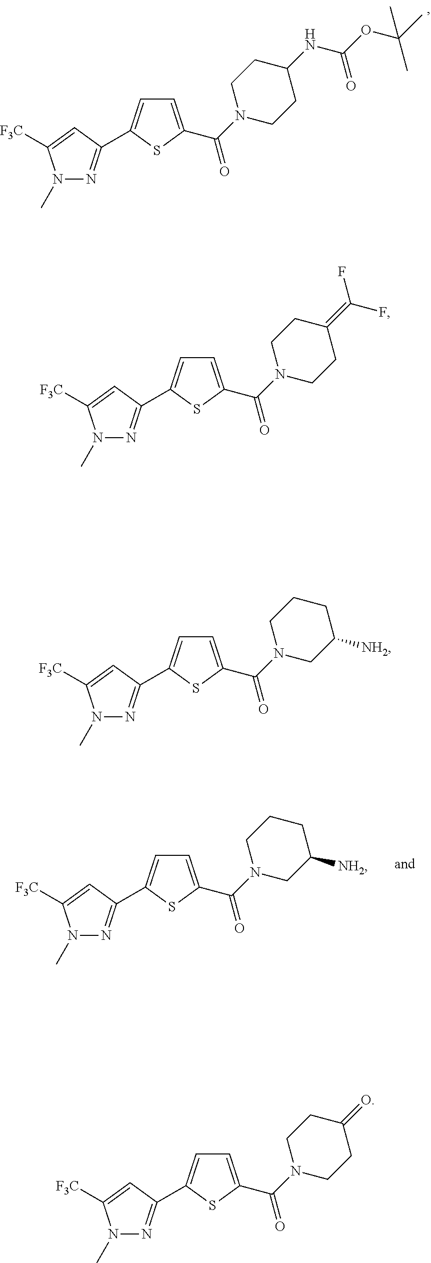 [SMILES: Cn1nc(-c2ccc(C(=O)N3CCC(NC(=O)OC(C)(C)C)CC3)s2)cc1C(F)(F)F ; Cn1nc(-c2ccc(C(=O)N3CCC(=C(F)F)CC3)s2)cc1C(F)(F)F ; Cn1nc(-c2ccc(C(=O)N3CCC[C@H](N)C3)s2)cc1C(F)(F)F ; Cn1nc(-c2ccc(C(=O)N3CCC[C@@H](N)C3)s2)cc1C(F)(F)F ; Cn1nc(-c2ccc(C(=O)N3CCC(=O)CC3)s2)cc1C(F)(F)F]

In some embodiments, the chemical entity can include one or more of the following: a compound of Formula (III), a pharmaceutically acceptable salt of a compound of Formula (III), a pharmaceutically acceptable solvate of a compound of Formula (III), a pharmaceutically acceptable conformer of a compound of Formula (III), a pharmaceutically acceptable prodrug of a compound of Formula (III), and a pharmaceutically acceptable crystalline form (or polymorph) of a compound or Formula (III).

In some embodiments, the chemical entity is a compound of Formula (III), a pharmaceutically acceptable salt of a compound of Formula (III), or a pharmaceutically acceptable prodrug of a compound of Formula (III).

In other embodiments, the chemical entity is a compound of Formula (III) or a pharmaceutically acceptable salt of a compound of Formula (III).

Additionally some MAOIs are selective and irreversible, while others are selective and reversible. In one embodiment, the MAO inhibitor, e.g., an inhibitor of Formula (I), Formula (II) or Formula (III), is a selective MAO-B inhibitor, such as a selective and reversible MAO-B inhibitor. In another aspect, the selective MAO-B inhibitor is an irreversible inhibitor.

### Compositions

In some embodiments compounds of Formula (I), Formula (II), or Formula (III), or derivatives thereof, are used, alone or in combination with one or more additional active ingredients, to formulate pharmaceutical compositions.

A pharmaceutical composition of the invention comprises: (a) an effective amount of at least one active agent in accordance with the invention; and (b) a pharmaceutically acceptable excipient.

### Formulations and Administration

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Osol, ed.), 1980, 1553-1593.

Any suitable route of administration can be employed for providing an animal, especially a human, with an effective dosage of a compound of the present invention. For example, oral, rectal, topical, parenteral, ocular, pulmonary, nasal, and the like can be employed. Dosage forms include tablets, troches, dispersions, suspensions, solutions, capsules, creams, ointments, aerosols, and the like.

Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent, or excipient used will depend upon the means and purpose for which the compound of the present invention is being applied. Solvents are generally selected based on solvents recognized by persons skilled in the art as safe (GRAS) to be administered to an animal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations can be prepared using conventional dissolution and mixing procedures. For example, the bulk drug substance (i.e., a compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable and appropriate dosage of the drug.

The pharmaceutical composition (or formulation) for application can be packaged in a variety of ways, depending upon the method used to administer the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label may also include appropriate warnings.

The active ingredient can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The present compounds can be systemically administered, e.g., orally, in combination with a pharmaceutically acceptable vehicle such as an inert diluent or an assimilable edible carrier. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations can, of course, be varied and can conveniently be between about 2 to about 60% of the weight of a given unit dosage form. The amount of active compound in such therapeutically useful compositions is such that an effective dosage level will be obtained.

### Dosage Forms

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are typically prepared by incorporating the active ingredient in the required amount in the appropriate solvent with a variety of the other ingredients enumerated above, as required, followed by filter sterilization. In the case of sterile powders for the preparation of sterile injectable solutions, common methods of preparation are vacuum drying and the freeze drying techniques, which yield a powder of the active ingredient plus any additional desired ingredient present in the previously sterile-filtered solutions.

For topical administration, the present active ingredients can be applied in pure form, i.e., when they are liquids. However, it will generally be desirable to administer them to the skin as compositions or formulations, in combination with a dermatologically acceptable carrier, which can be a solid or a liquid.

Useful solid carriers include finely divided solids such as talc, clay, microcrystalline cellulose, silica, alumina, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user

In other embodiments, topical, intravenous, intraperitoneal or other parenteral administration is excluded as a method of administering the instantly disclosed compositions. In these embodiments, the instantly disclosed compositions are instead exclusively administered orally.

### Methods and Uses

The present invention provides therapeutic methods using a chemical entity of the present invention, such as compounds corresponding to known MAO-B inhibitors known in the art, and more particularly, those of Formula (I), Formula (II), or Formula (III), whether alone or in combination.

### Neurological Disorders and Deficits

As described in more detail herein, therapeutic methods include methods of treating a neurological disorder, and more particularly, treating a neurological disorder during rehabilitation. In some embodiments, the methods are directed to treating a neurological disorder, comprising administering to a subject in need thereof an effective amount of an MAO-B inhibitor during rehabilitation. In some embodiments, the methods are directed to treating a neurological disorder during rehabilitation, comprising: (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone).

In particular embodiments of these methods, treating is directed to a neurological deficit associated with the neurological disorder. Neurological deficits impacted by trauma related disorders (and which can be targeted during rehabilitation) include impairments in cognitive and motor functions. Cognitive function impairments, for example, can manifest as deficits in understanding speech or writing (aphasia); knowing the right words but having trouble saying them clearly (dysarthria); as well as deficits in other cognitive functions, such as attention, reasoning, planning, execution, and learning and memory. Motor function impairments, for example, can be manifested by deficits in upper and lower extremity function; by problems with balance or coordination; by deficits in gross motor skills such as gait and walking speed; and by deficits in fine motor skills or manual dexterity.

In some embodiments, the neurological deficit is a motor deficit (or impairment). Motor function impairments can manifest, for example, as weakness or paralysis, deficits in upper and lower extremity function, problems with balance or coordination; impairments of gross motor skills; and deficits in fine motor skills.

In some embodiments, the neurological deficit is a cognitive deficit (or impairment). Cognitive function impairments can manifest, for example, as deficits in attention (e.g., sustained attention, divided attention, selective attention, processing speed), executive function (e.g., planning, decision, and working memory); memory (e.g., immediate memory; recent memory, including free recall, cued recall, and recognition memory, and long-term memory). Long-term memory can be divided into (a) explicit (declarative memory) memory, such as episodic, semantic, and autobiographical memory, and (b) implicit memory (procedural memory). Cognitive impairments can also be manifested in use (or lack of use) of expressive language, including naming, word recall, fluency, grammar, and syntax; understanding speech or writing (e.g., aphasia); perceptual-motor functions (e.g., abilities encompassed under visual perception, visual-constructional, perceptual-motor praxis, and gnosis); and social cognition (e.g., recognition of emotions, theory of mind).

In some embodiments, the cognitive deficit is a deficit in memory formation and more particularly, is a deficit in long-term memory.

Neurotrauma

In some embodiments, the neurological disorder is a “neurotrauma disorder” (or “neurotrauma”). Accordingly, in some embodiments, the methods involve treating a neurological deficit associated with a neurotrauma disorder, comprising administering to a subject in need thereof an effective amount of an MAO-B inhibitor during rehabilitation from the neurotrauma.

In other embodiments, the methods involve treating a neurological deficit during rehabilitation of a neurotrauma disorder, comprising: (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone).

A neurotrauma includes, but is not limited to: (i) vascular diseases due to stroke (e.g., ischemic stroke or hemorrhagic stroke) or ischemia; (ii) microvascular disease arising from diabetes or arthrosclerosis; (3) traumatic brain injury (TBI), which includes penetrating head injuries and closed head injuries; (4) tumors, such as nervous system cancers, including cerebral tumors affecting the thalamic or temporal lobe; (5) hypoxia; (6) viral infection (e.g., encephalitis); (7) excitotoxicity; and (8) seizures.

In specific embodiments, the neurotrauma disorder is selected from the group consisting of a stroke, a traumatic brain injury (TBI), a head trauma, and a head injury. In some embodiments, the neurotrauma disorder is stroke. In some embodiments, the neurotrauma disorder is TBI.

Muscle Disorders

In some embodiments, the neurological disorder is a muscle disorder, which includes a muscle weakness and muscle paralysis. In a particular aspect, the muscle disorder is a non-stroke muscle disorder, which includes a non-stroke muscle weakness and non-stroke muscle paralysis. Accordingly, in some embodiments, the methods involve treating a neurological deficit associated with a muscle disorder (or non-stroke muscle disorder), comprising administering to a subject in need thereof an effective amount of an MAO-B inhibitor during rehabilitation of the disorder.

In other embodiments, the methods involve treating a neurological deficit during rehabilitation of a muscle (or non-stroke muscle) disorder, comprising: (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance is improved compared to that produced by training alone).

In some embodiments, the non-stroke muscle disorder is muscle paralysis—a loss of muscle function in one or more muscles. Paralysis can be accompanied by a loss of feeling (sensory loss) in the affected area. Paralysis is most often caused by damage in the nervous system, especially the spinal cord.

Other major non-stroke causes of a muscle weakness or paralysis are trauma with nerve injury, a viral infection, poliomyelitis, cerebral palsy, peripheral neuropathy, Parkinson's disease, ALS, botulism, spina bifida, demyelinating disease, multiple sclerosis, and Guillain-Barrd syndrome. Paralysis can occur in localized or generalized forms, or it may follow a certain pattern. Most paralyses caused by nervous-system damage (e.g., spinal-cord injuries) are constant in nature.

In some embodiments, the non-stroke muscle disorder is Bell's palsy, which is characterized by the appearance of a one sided facial droop within about 72 hours. This facial paralysis results from a dysfunction of the facial nerve, causing an inability to control facial muscles on the affected side. The facial nerves control a number of functions, such as blinking and closing the eyes, smiling, frowning, lacrimation, salivation, flaring nostrils and raising eyebrows. (Facial paralysis is called Bell's palsy when there is no identification of a specific cause, such as a brain tumor, stroke, myasthenia gravis, or Lyme disease.)

In some methods, the MAO-B inhibitor is administered chronically. In some embodiments, rehabilitation includes physical therapy, occupational therapy, or cognitive therapy.

In one aspect, treatment is directed to facilitating muscle re-education (which is described in greater detail herein) in a patient during rehabilitation of a muscle disorder, and more particularly, a non-stroke muscle disorder. In another aspect, treatment is directed to increasing the range of motion in a patient during re-education of a muscle disorder, and more particularly, a non-stroke muscle disorder.

### Subjects

In some embodiments, the subject being treated by methods of the present invention is a human patient. In some embodiments, the human patient is a post-acute trauma patient, meaning that the patient has been medically stabilized from the initial trauma (e.g., neurotrauma disorder, such as stroke or TBI).

Rehabilitation provided to such a post-acute trauma patient can therefore be referred to as “post-acute trauma rehabilitation.”

In other particular embodiments, the human patient is a post-acute stroke patient, meaning that the patient has been medically stabilized from the stroke, and the rehabilitation is “post-acute stroke rehabilitation.” In such embodiments, the stroke patient is no longer in the acute-stage of stroke care.

In other embodiments, the subject is a post-acute TBI patient, and the rehabilitation is post-acute TBI rehabilitation.

In some embodiments, the subject is in a standard rehabilitation programs under the supervision of therapists prior to drug treatment. In one aspect, the subject is provided with cognitive, motor, or occupational training as part of rehabilitation. In another aspect, the subject is provided with muscle (neuromuscular) re-education training.

### Inhibitors and Administration

The present embodiments provide for a use of a composition of any of the embodiments and examples disclosed herein, or a pharmaceutically acceptable salt or prodrug ester of the active ingredient thereof, for the manufacture of a medicament useful for rehabilitating a patient from a neurotrauma disorder. The medicament can be labeled (indicated) for long-term, i.e., chronic use after the patient has suffered the neurotrauma (e.g., stroke), as described herein.

The chemical details of exemplary MAO-B inhibitors are described in more detail elsewhere in this specification. In one embodiment, the active ingredient is selective for MAO-B over MAO-A when administered to a human. In specific embodiments of the invention, the active ingredient inhibits the activity of MAO-B 2 times, 5 times, 10 times, 25 times, 50 times, 100 times, 200 times, 500 times, 1000 times, 2000 times, or 4000 times more than it inhibits the activity of MAO-A.

In one embodiment, non-selective MAO-B inhibitors are excluded as an active ingredient, particularly in embodiments in which the active agent is used in methods of rehabilitating a patient from a stroke. In one embodiment, MAO-B inhibitors that inhibit MAO-A to an appreciable extent are excluded as an active ingredient, particularly in embodiments in which the active agent is used in methods of rehabilitating a patient from a stroke.

Chemical entities of the present invention can be administered as a monotherapy or as part of a combination therapy. “Monotherapy” refers to a treatment regimen based on the delivery of one therapeutically effective compound, whether administered as a single dose or several doses over time.

In another aspect, one or more of the compounds (or salts, prodrugs, or metabolites thereof) of the present invention can be co-administered or used in combination with one or more additional therapies known in the art. For example, compounds of the present invention (and derivatives thereof) can be used as adjunct therapy with dopamine preparations, dopamine agonists, or COMT agents (drugs that inhibit the action of catechol-methyl transferase) for the treatment of Parkinson's disease. Compounds (and derivatives thereof) of the present invention can also be combined with other treatments or modalities, such as surgery, medical devices, or feedback systems.

In some embodiments, the MAO-B inhibitor is a reversible inhibitor. In some embodiments, the MAO-B inhibitor is not selegiline. In some embodiments, the MAO-B inhibitor is not a prodrug of an amphetamine. In other embodiments, metabolism of the MAO-B inhibitor in the subject does not produce an amphetamine, such as methamphetamine. In this regard, selegiline is partly metabolized in vivo to L-methamphetamine, one of the two enantiomers of methamphetamine. (Engberg et al., J. Pharmacol. Exp. Ther. 1991, 259, 841-847).

### Timing and Duration

In some embodiments, the administering step occurs in conjunction with training. By “in conjunction” is meant that the active ingredient (e.g., MAO-B inhibitor) enhances CREB pathway function during training. In some embodiments, the active ingredient is administered before each training sessions. In some embodiments, the active ingredient is administered before and/or during each training session.

In some embodiments, the MAO-B inhibitor is administered to the subject during the acute stage and during the post-acute stage. In some embodiments, the MAO-B inhibitor is administered to the subject only after the acute stage has ended, i.e., only during the post-acute stage.

In some embodiments, the MAO-B inhibitor is administered chronically, meaning that it is indicated for long-term use, such as long-term use after the acute stage of the neurotrauma disorder, such as stroke or TBI, has ended and the patient has been medically stabilized. Such long-term use can include the period of a post-trauma rehabilitation program. Duration of administration can further extend beyond the duration of the formal rehabilitation program. For example, such post-formal rehabilitation period can encompass a period of 3 months, 6, months, 12 months, or longer, following the end of a rehabilitation program.

One aspect of the present invention provides a method of treating a patient who is rehabilitating from a stroke. Embodiments of this method include administering to the patient a composition that includes a MAO-B inhibitor active ingredient, such as a reversible MAO-B inhibitor active ingredient. For example, the MAO-B inhibitor can be administered beginning shortly after a subject suffers a stroke, or can be administered during and in conjunction with a formal rehabilitation program.

For example, one embodiment provides administering to the patient undergoing rehabilitation a composition that includes a MAO-B inhibitor active ingredient, such as a reversible MAO-B inhibitor active ingredient, in which the administration occurs prior to, and within three hours, within two hours, or within one hour of the initiation of the particular rehabilitation regimen for that patient, for that particular day. In one embodiment, the MAO-B inhibitor active ingredient, such as a reversible MAO-B inhibitor active ingredient, is exclusively administered prior to, and within three hours, within two hours, or within one hour of the initiation of the particular rehabilitation session for that patient, for that particular day.

The particular rehabilitation program employed is not limited, and includes any cognitive or motor training, and particularly post-stroke rehabilitation and TBI rehabilitation as described in greater detail below.

Thus, the timing of administration can be specified to occur prior to, and within a period time before the initiation of a scheduled stroke rehabilitation session for that patient. For example, if the patient is scheduled to undergo stroke rehabilitation on Monday, Wednesday and Friday, administration of the instant compositions can occur on Monday, Wednesday and Friday within three hours, within two hours or within one hour of the initiation of such session. Instructions can be provided to the patient to administer the composition prior to going to the rehabilitation clinic, or the composition can be administered at the rehabilitation clinic just prior to the initiation of the scheduled rehabilitation session (i.e., at the clinic intake or waiting room, or any other time before the substantive rehabilitation begins).

An MAO-B inhibitor can also be administered as part of maintenance program after a patient has completed a rehabilitation program. For example, it can be administered to a subject at home. In some embodiments, administration during a maintenance program can be of relative short duration—less than one year after the initial trauma event, or the duration can be more than one year after the initial neurotrauma disorder event.

In some embodiments, the inhibitor can be administered in regular intervals over a long term period (e.g., at least over a few weeks, but generally for at least a month, year, or for the remaining lifetime of the subject). Throughout this long term period, the instantly disclosed compositions can be administered, for example, once weekly, twice weekly, thrice weekly, every other day, daily, twice daily, thrice daily, four times a day, five times a day, or six times a day.

### Dosing

In general a suitable dose of active ingredient for use in the presently disclosed methods can be determined by an artisan of ordinary skill, for example, by comparing their in vitro activity and in vivo activity in animal models. Methods for the extrapolation of effective dosages in mice, and other animals, to humans are known in the art (see, e.g., U.S. Pat. No. 4,983,949). Useful dosages of particular MAO-B inhibitors disclosed herein are also known in the art. (see, e.g., U.S. Published Application No. 2014/0275548, U.S. Pat. No. 8,222,243, and U.S. Pat. No. 8,399,487, each of which being hereby incorporated by reference in their entirety).

While not being limited thereto, a suitable dose will often be, for example, in the range of from about 0.15 to about 100 mg/kg, e.g., from about 0.75 to about 75 mg/kg of body weight per day, or 1 to about 50 mg/kg of body weight per day.

The active ingredient is conveniently administered as a pharmaceutical composition in unit dosage form; for example, containing 1 to 1000 mg, conveniently 5 to 100 mg, most conveniently, 25 to 100 mg of active ingredient per unit dosage form.

The desired dose can conveniently be presented in a single dose or as divided doses administered at appropriate intervals, for example, as two, three, four or more sub-doses per day. The sub-dose itself can be further divided, e.g., into a number of discrete loosely spaced administrations

### Rehabilitation and Training

In some embodiments, rehabilitation in the therapeutic methods herein is post-acute trauma rehabilitation, and more particularly, is post-acute stroke rehabilitation or post-acute TBI rehabilitation.

Chemical entities and compositions can be administered with training to treat cognitive or motor deficits associated with neurological disorders, as described in more detail herein. In certain embodiments, a composition containing an effective amount of MAO-B inhibitor active ingredient, such as the instantly disclosed MAO-B inhibitors, is used in methods to enhance the efficiency of cognitive or motor training (collectively “training”) during rehabilitation.

In some embodiments, training comprises a battery of tasks directed to the neurological function. In some embodiments, the training is part of physical therapy, cognitive therapy, or occupational therapy.

Training generally requires multiple sessions to attain the desired benefits, for example, to rehabilitate a motor deficit or language deficit following stroke. This can be costly and time-consuming, deterring subject compliance and the realization of real world benefits that endure over time. The efficiency of such training protocols can be improved by administering the instantly disclosed pharmaceutical compositions containing an effective amount of an MAO-B inhibitor, e.g., a reversible MAO-B inhibitor, such as MAO-B inhibitors of Formula (I), (II), or (III), described in U.S. Published Application No. 2014/0275548, U.S. Pat. No. 8,222,243, and U.S. Pat. No. 8,399,487, respectively.

The resulting improvement in efficiency of any methods disclosed herein can be manifested in several ways, for example, by enhancing the rate of recovery, or by enhancing the level of recovery. Accordingly, in one aspect, administering an MAO-B inhibitor of the present invention with a training protocol can decrease the amount of training sufficient to improve performance of a neurological function compared with training alone. In another aspect, administering an MAO-B inhibitor with a training protocol may increase the level of performance of a neurological function compared to that produced by training alone.

In general, a training protocol (or module) comprises a set of distinct exercises that can be process-specific or skill-based: Process-specific training focuses on improving a particular domain such as attention, memory, language, executive function, or motor function. Here one goal of training is to obtain a general improvement that transfers from the trained activities to untrained activities associated with the same cognitive or motor function or domain. For example, an auditory cognitive training protocol can be used to treat a subject with impaired auditory attention after suffering from a stroke. At the end of training, the subject should show a generalized improvement in auditory attention, manifested by an increased ability to attend to and concentrate on verbal information.

Skill-based training is aimed at improving performance of a particular activity or ability. Here the goal of training is to obtain a general improvement in the skill or ability. The different exercises within such a protocol will focus on core components underlying the skill. Modules for increasing memory, for example, can include tasks directed to the recognition and use of fact, and the acquisition and comprehension of explicit knowledge rules.

Some rehabilitation programs can rely on a single strategy (such as computer-assisted cognitive training) targeting either an isolated cognitive function or multiple functions concurrently. For example, the CogState testing method comprises a customizable range of computerized cognitive tasks able to measure baseline and change in cognitive domains underlying attention, memory, executive function, as well as language and social-emotional cognition (e.g., Yoshida et al., PloS ONE 2011, 6, e20469; Frederickson et al., Neuroepidemiology 2010, 34, 65-75). Other rehabilitation programs can use an integrated or interdisciplinary approach.

Cognitive and motor training programs can involve computer games, handheld game devices, and interactive exercises.

Cognitive and motor training programs can also employ feedback and adaptive models. Some training systems, for example, use an analog tone as feedback for modifying muscle activity in a region of paralysis, such as facial muscles affected by Bell's palsy. (e.g., Jankel, Arch. Phys. Med. Rehabil. 1978, 59, 240-242.). Other systems employ a feedback-based close loop system to facilitate muscle re-education or to maintain or increase range of motion. (e.g., Stein, Expert Rev. Med. Devices 2009, 6, 15-19.)

Muscle Re-Education

Muscle re-education (or “neuromuscular re-education”) is a general term that refers to techniques that attempt to retrain the neuromuscular system to function properly. Muscle movement patterns are affected when nerves or muscles experience damage or injury. This can result from trauma, medical conditions, and neurological conditions, such as stroke and traumatic brain injury. Neuromuscular re-education is used by rehabilitation or occupational therapists to facilitate the return of normal movement in persons with neuromuscular impairments. The general aim is either to reestablish normal patterns of movement in injured people or to create normal patterns of movement in disabled people, by practicing a variety of exercises. Neuromuscular re-education can also be directed to improving balance, coordination, posture, and proprioception. A neuromuscular re-education program may consist, for example, of repetitive movements, posturing, and stimulation designed to reinforce nerve signals for functional movements.

In some embodiments, a composition containing an effective amount of MAO-B inhibitor active ingredient, such as the instantly disclosed MAO-B inhibitors, is used in methods to facilitate muscle re-education during rehabilitation. In particular embodiments, these methods are used to maintain (or increase) range of motion, and more particularly, can be used with feedback systems. These methods of muscle re-education can therefore be applied to any neurological disorder disclosed herein, including neurological deficits associated with any neurological disorder, such as a neurotrauma, stroke, TBI, a muscle disorder, and a non-stroke muscle disorder.

Accordingly, the present invention provides methods of facilitating muscle re-education, comprising administering to a subject in need thereof an effective amount of an MAO-B inhibitor during rehabilitation of a neurological disorder. In some embodiments, the methods are directed to facilitating muscle re-education during rehabilitation of a muscle disorder, comprising: (a) administering to a subject in need thereof an effective amount of an MAO-B inhibitor; (b) providing muscle re-education training under conditions sufficient to improve performance of a muscle function whose impairment is associated with the disorder; and (c) repeating steps (a) and (b) one or more times, whereby the amount of training sufficient to improve said performance is reduced compared to that produced by training alone (or whereby the performance is improved compared to that produced by training alone). In some embodiments, the neurological disorder is a neurotrauma, such as stroke and TBI. In other embodiments, the neurological disorder is a muscle disorder. In one aspect the muscle disorder is muscle weakness or muscle paralysis. In another aspect, the muscle disorder is a non-stroke muscle disorder, such as non-stroke muscle disorder or non-stroke muscle paralysis.

### Stroke Rehabilitation

In some embodiments, chemical entities (i.e., active ingredients) and compositions of the present invention are useful in treating stroke, and in more specific embodiments, treating motor or cognitive impairments during post-stroke rehabilitation.

Stroke is the fourth leading cause of death in the United States and the leading cause of serious, long-term disability. (Kochanek et al., Nat Vital Stat Rep. 2011, 60, 1-117; Go et al., Circulation 2012, e2-e241). A stroke is a sudden interruption of blood flow to the brain, and includes, but is not limited to, hemorrhagic and ischemic strokes. Hemorrhagic strokes, which are caused by bleeding in the brain, typically result from a ruptured blood vessel. Ischemic Strokes account for the vast majority of all strokes and are caused by a loss of blood flow to an area of the brain, such as that resulting from a blood clot lodged in an artery to a portion of the brain.

Stroke care is a temporal continuum that includes acute (immediate), subacute, and chronic phases. Any stroke care commencing after the acute phase (i.e., after a patient is medically stabilized) is considered a post-acute stroke setting. Although time frames can vary among institutions and under different circumstances, the acute phase is about 24 hours from stroke, the subacute phase is from 24 hours to about 2 weeks, and the chronic is beyond the subacute phase (e.g., after about 2 weeks). Treatments during the acute phase are known, and can directly target the initial damage triggered by the stroke; they usually involve using agents to dissolve clots and restore blood flow to reduce tissue damage and stabilize the patient. The efficacy of acute treatments is typically limited to a short time window spanning only a few hours from stroke onset.

Subacute stroke treatments, which typically begin in the hospital, focus on managing medical complications, such as cerebral edema, seizures, hemorrhagic conversion of an ischemic stroke, evolution of neurologic defects, infection, and delirium. (e.g., Bernheisel et al., Am. Fam. Physician 2001, 84, 1383-1388).

After the patient has been medically stabilized, stroke treatment generally shifts to rehabilitation in the chronic phase. Stroke rehabilitation during the chronic phase involves training protocols and other therapeutic modalities directed to cognitive and motor deficits that persist after the initial stroke injury, the goal being to restore and recover neurological function as much as possible to enable people with post-stroke disabilities to reach and maintain optimal physical, intellectual, psychological and social function (Quinn et al., J. Rehabil. Med. 2009, 41, 99-111).

One embodiment of the present invention provides a method of treating motor impairments associated with a stroke in a subject undergoing motor rehabilitation in a post-acute stroke setting, comprising administering to the subject a MAO-B inhibitor (e.g., a reversible MAO-B inhibitor, such as, but not limited to, the reversible MAO-B inhibitors disclosed herein). Another embodiment of the present invention provides a method of treating functional impairments associated with a stroke in a subject undergoing functional rehabilitation in a post-acute stroke setting, comprising administering to the subject a MAO-B inhibitor (e.g., a reversible MAO-B inhibitor, such as, but not limited to, the reversible MAO-B inhibitors disclosed herein). The compositions can be administered more than once, and the compositions can be administered on a regular basis. For example, the compositions can be administered at least daily, or at least weekly. In certain embodiments, the first administration of a composition including a MAO-B inhibitor is no earlier than: 1 day after onset of the stroke, 2 days after onset of the stroke, 4 days after onset of the stroke, 1 week after onset of the stroke, 1 month after onset of the stroke, 2 months after onset of the stroke, 6 months after onset of the stroke, or 1 year after onset of the stroke.

Post-stroke rehabilitation in a post-acute stroke setting (i.e., post-acute stroke rehabilitation) encompasses a wide range of activities, in addition to standard medical care, and can occur in multiple environments, such as a rehabilitation hospital, long-term care facility, outpatient clinic, or at home. Post-acute stroke rehabilitation is typically a comprehensive program coordinated by a team of medical professionals. A physical therapist on the team, for example, can focus on maintaining and restoring range of motion and strength in affected limbs, maximizing mobility in walking, improving manual dexterity, and rehabilitating other motor and sensorimotor functions. A mental health professional, such as a neuropsychologist, can be involved in the treatment of loss of cognitive skills. An occupational therapist can assess and provide training in cognitive skills related to the individual's ability to perform activities of daily living. Such activities can include dressing, bathing, grooming, and eating. Depending on the center, occupational therapists can also evaluate the patient's thinking skills, such as orientation, memory, attention, concentration, calculation, problem-solving, reasoning and judgment; assess visual problems in the patient; and help the patient manage more complex activities such as meal preparation/cooking, money management, and getting involved in community activities.

Neurological functions impacted by stroke (and which can be targeted during rehabilitation) include impairments in cognitive and motor functions. Cognitive function impairments, for example, can manifest as deficits in understanding speech or writing (aphasia); knowing the right words but having trouble saying them clearly (dysarthria); as well as deficits in other cognitive functions, such as attention, reasoning, planning, execution, and learning and memory. Motor function impairments, for example, can manifest as weakness (hemiparesis) or paralysis (hemiplegia) on one side of the body that may affect the whole side or just the arm or leg; by problems with balance or coordination; disruption of gross motor skills such as gait and walking speed; deficits in fine motor skills or manual dexterity; and deficits in upper and lower extremity function. In particular embodiments, motor function impairments are upper extremity motor impairments, and in particular, mild to moderate upper extremity motor impairments.

Accordingly, the present invention provides the use of a MAO-B inhibitor in the treatment of stroke, including in conjunction with post-stroke rehabilitation, particularly post-acute stroke rehabilitation. In certain embodiments, chemical entities of the present invention are useful during such stroke rehabilitation to treat stroke deficits (or “post-stroke deficits”) resulting from impaired neurological functions.

In certain embodiments, the first initial dosage of MAO-B inhibitor in the treatment of stroke (e.g., post-stroke rehabilitation or post-acute stroke rehabilitation) occurs no earlier than: about 1 day after onset of the stroke, about 2 days after onset of the stroke, about 4 days after onset of the stroke, about 1 week after onset of the stroke, about 1 month after onset of the stroke, about 2 months after onset of the stroke, about 6 months after onset of the stroke, or 1 year after onset of the stroke. In other embodiments, the first initial dosage of MAO-B inhibitor, in the treatment of stroke, is in patients who are within about 2 days, about 1 week, about 1 month, about 2 months, about 6 months, about 1 year, or more than 1 year of onset of the stroke.

In some embodiments, the methods are directed to treating a neurological deficit associated with a stroke, comprising administering to a subject in need thereof an effective amount of an MAO-B inhibitor during rehabilitation. In some embodiments, the methods are directed to treating a neurological disorder during rehabilitation, comprising: (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone (or whereby the performance of the neurological function is improved compared to that produced by training alone).

In one aspect the neurological deficit is a motor deficit, and the training is motor training. In another aspect, the neurological deficit is a cognitive deficit, and the training is cognitive training.

In one aspect, the MAO-B inhibitor is a chemical entity of the present invention. In some embodiments, the deficit is a motor deficit. In other embodiments, the deficit is a cognitive deficit, particularly, a deficit in memory formation, and more specifically, a deficit in long-term memory formation. In still other embodiments, the deficit can include a cognitive and motor deficit. In another aspect, training comprises a battery of tasks directed to the neurological function. In a specific aspect, the reduction in the amount of training is a reduction in the number of training sessions.

In one aspect, the subject is a human. In a further embodiment, the administering step is in conjunction with the training step. In some embodiments, the MAO-B inhibitor is administered before each training session. In some embodiments, the inhibitor is administered before each training session. In other aspects, the compound is administered before and/or during each training session.

In some embodiments, the invention provides methods of treating a cognitive disorder in a patient who has suffered a stroke, particularly patients who have recently suffered a stroke, comprising treating the animal with a presently disclosed composition containing an effective amount of a MAO-B inhibitor in conjunction with cognitive training.

In one aspect, the method comprises: (a) providing motor training to a subject in need of treatment of a motor deficit under conditions sufficient to produce an improvement in performance by said animal of a motor function whose impairment is associated with said cognitive deficit, particularly a cognitive defect in a patient who has suffered a stroke; (b) administering a composition of the present invention to the animal in conjunction with said motor training; (c) repeating steps (a) and (b) one or more times; and (d) reducing the number of training sessions sufficient to produce the improvement in performance, relative to the same improvement in performance produced by motor training alone.

In another aspect, the method comprises: (a) providing motor training to a subject in need of treatment of a motor deficit under conditions sufficient to produce an improvement in performance by said animal of a motor function whose impairment is associated with said cognitive deficit, particularly a cognitive defect in a patient who has suffered a stroke; (b) administering a composition of the present invention to the animal in conjunction with said motor training; (c) repeating steps (a) and (b) one or more times; and (d) producing a long-lasting improvement in performance of said function relative to the improvement in performance of said function produced by motor training alone (or whereby the performance is improved compared to that produced by training alone).

In some embodiments, the stroke is hemorrhagic stroke. In other embodiments, the stroke is an ischemic stroke.

The presently disclosed compositions can be administered before, during or after one or more of the training sessions. In a particular embodiment, a presently disclosed composition is administered before and/or during each training session. Treatment with an augmenting agent in connection with each training session is also referred to as the “augmenting treatment”.

Training protocols are employed in rehabilitating individuals who have some form and degree of cognitive or motor dysfunction resulting from a stroke. Because multiple training sessions are often required before an improvement or enhancement of a specific aspect of cognitive (or motor) performance (ability or function) is obtained in the individuals, training protocols are often very costly and time-consuming. Augmented training methods that employ the presently disclosed compositions are more efficacious and therefore more cost-effective.

Once a patient is stabilized following a stroke the standard of care dictates extensive motor or cognitive rehabilitation. During this rehabilitation the patient often regains lost skills, finally resulting in improved functional outcome. It would be beneficial if pharmaceutical treatments could be developed to enhance motor or cognitive rehabilitation following TBI, and thus improve functional outcome.

Cognitive and motor training protocols and the underlying principles are known in the art (e.g., Jaeggi et al., Proc. Natl. Acad. Sci. USA 2011, 108, 10081-10086; Chein et al., Psychon. Bull. Rev. 2010, 17, 193-199; Klingberg, Trends Cogn. Sci. 2010, 14, 317-324; Owen et al., Nature 2010, 465, 775-778; Tsao et al., J. Pain 2010, 11, 1120-1128; Lustig et al., Neuropsychol. Rev. 2009, 19, 504-522; Park and Reuter-Lorenz, Ann. Rev. Psych. 2009, 60, 173-196; Oujamaa et al., Ann. Phys. Rehabil. Med. 2009, 52, 269-293; Frazzitta et al., Movement Disorders 2009, 8, 1139-1143; Jaeggi et al., Proc. Natl. Acad. Sci. USA 2008, 105, 6829-6833; Volpe et al., Neurorehabil. Neural Repair 2008, 22, 305-310; Fischer et al., Top. Stroke Rehab. 2007, 14, 1-12; Jonsdottir et al., Neurorehabil. Neural Repair 2007, 21, 191-194; Stewart et al., J. Neurol. Sci. 2006, 244, 89-95; Krakauer, Curr. Opin. Neurol. 2006, 19, 84-90; Belleville et al., Dement. Geriatr. Cogn. Disord. 2006, 22, 486-499; and Klingberg et al., J. Am. Acad. Child. Adolesc. Psychiatry 2005, 44, 177-186).

In some embodiments, the protocols can be used to treat, or rehabilitate, cognitive or motor impairments in subjects who have suffered a stroke. Such protocols can be restorative or remedial, intended to reestablish prior skills and functions, or they can be focused on delaying or slowing cognitive or motor decline due to the stroke and general aging. Other protocols can be compensatory, providing a means to adapt to a cognitive or motor deficit by enhancing function of related and uninvolved brain domains. In one aspect, the protocol is directed to facilitating muscle re-education in a post-stroke patient (e.g., a post-acute stroke patient). In another aspect the method is directed to increasing the range of motion in a post-stroke patient (e.g., a post-acute stroke patient).

### TBI Rehabilitation

In some embodiments, the neurotrauma disorder is a traumatic brain injury (TBI). More than 1.5 million TBIs occur each year in the US, with 125,000 of these resulting in permanent disability. Moreover, TBI is the leading cause of military casualties in the field and a leading source of long-term rehabilitation problems suffered by veterans. When not fatal (22% of moderate and 35% of severe TBI patients die within the first year following injury), TBI can result in permanent and severe physical, cognitive, and behavioral impairments, leaving sufferers in need of long term healthcare. Improved interventions during rehabilitation are therefore important to optimize functional recovery in post-TBI subjects, and more particularly, in post-acute TBI subjects.

TBI can be divided into two main classes of injuries: Penetrating Injuries and Closed Head Injuries. In Penetrating Injuries, a foreign object (e.g., a bullet or shrapnel from an explosive) enters the brain. Focal, or localized, damage occurs in specific regions along the route the object has traveled in the brain. Symptoms vary depending on the part of the brain that is damaged.

Closed Head Injuries involve a blow to the head, such as that occurring when one's head strikes the windshield or dashboard in a car accident, or when one one's head has a concussive impact in a contact sport such as football. Closed Head Injuries also particular blast injuries resulting in shockwave transmission through the skull and brain.

In some embodiments, chemical entities and compositions of the present invention are useful in treating traumatic brain injury (TBI), and in more specific embodiments, treating motor or cognitive impairments during post-TBI rehabilitation, and more particularly, during post-acute TBI rehabilitation.

Like stroke care, TBI case is a temporal continuum that includes immediate (acute) treatments to medically stabilize the patient from the trauma, and subsequent rehabilitative therapy (post-acute).

Methods of stabilizing a patient who has suffered a TBI are known. Typically, individuals identified as having experienced a TBI are seen in an emergency department and, along with a clinical exam, may undergo immediate neuro-radiological examination such as head computerized tomographic (CT) scan or magnetic resonance imaging (MRI), to assess signs of brain trauma (bleeding within the skull or brain known as intracranial hemorrhaging, increased pressure on the brain, or bruising of the brain known as contusions). Individuals with identified brain pathology are typically admitted to the hospital intensive care unit for close observation and needed medical interventions until the patient is medically stabilized. If the pressure on the brain becomes severe enough, surgical intervention to relieve this pressure becomes necessary. In less severe cases, the patient may be treated with medications to prevent medical complications associated with brain trauma and is closely monitored.

Accordingly, the present invention provides the use of an MAO-B inhibitor in the treatment of TBI, including during TBI rehabilitation (e.g., post-acute TBI rehabilitation), to treat TBI deficits (or “post-TBI deficits”) resulting from impaired neurological functions. In some embodiments, the present invention provides methods of treating a neurological deficit during post-acute TBI rehabilitation comprising: (a) administering to a subject in need thereof a MAO-B inhibitor during recovery of the subject from TBI; (b) providing training to the subject under conditions sufficient to improve performance of a neurological function whose impairment is due to the deficit; and (c) repeating steps (a) and (b) one or more times, whereby the amount of training sufficient to improve the performance is reduced compared to that produced by training alone (or whereby the performance is improved compared to that produced by training alone).

In certain embodiments, the first initial dosage of MAO-B inhibitor in the treatment of TBI (e.g., post-acute TBI rehabilitation) occurs no earlier than: about 2 days after onset of the TBI, about 4 days after onset of the TBI, about 1 week after onset of the TBI, about 1 month after onset of the TBI, about 2 months after onset of the TBI, about 6 months after onset of the TBI, or about 1 year after onset of the TBI. In other embodiments, the first initial dosage of MAO-B inhibitor in the treatment of TBI is in patients who are within about 2 days, about 1 week, about 1 month, about 2 months, about 6 months, about 1 year, or more than 1 year of onset of the TBI.

In one aspect, the MAO-B inhibitor is a chemical entity of the present invention. In some embodiments, the deficit is a motor deficit. In other embodiments, the deficit is a cognitive deficit, particularly, a deficit in memory formation, and more specifically, a deficit in long-term memory formation. In still other embodiments, the deficit may include a cognitive and motor deficit. In another aspect, training comprises a battery of tasks directed to the neurological function. In a specific aspect, the reduction in the amount of training is a reduction in the number of training sessions.

In one aspect, the subject is a human. In a further embodiment, the administering step is in conjunction with the training step. In some embodiments, the MAO-B inhibitor is administered before each training session. In some embodiments, the inhibitor is administered before and/or during each training session. In one aspect, treatment is directed to facilitating muscle re-education in a post-TBI patient, and more particularly, in a post-acute TBI patient. In another aspect, treatment is directed to increasing the range of motion in a post-TBI patient, and more particularly, in a post-acute TBI patient.

The present disclosure will be further illustrated by the following non-limiting Examples. These Examples are understood to be exemplary only, and they are not to be construed as limiting the scope of the invention as defined by the appended claims.

### EXAMPLES

### Example 1: MAO-B Inhibitor Compositions

Tablets and capsules for oral administration containing the following unit dosage amounts can be prepared by conventional procedures well known in the pharmaceutical arts. Compound X is any one of the instantly disclosed active ingredients.

Tablet 1

| Component | mg/tablet |
|---|---|
| Compound X (MAO-B inhibitor) | 100 |
| Lactose | 77.5 |
| Povidone | 15.0 |
| Croscarmellose sodium | 12.0 |
| Microcrystalline cellulose | 92.5 |
| Magnesium stearate | 3.0 |
| Total | 300 |

Tablet 2

| Component | mg/tablet |
|---|---|
| Compound X (active ingredient) | 50 |
| Microcrystalline cellulose | 410.0 |
| Starch | 50.0 |
| Sodium starch glycolate | 15.0 |
| Magnesium stearate | 5.0 |
| Total | 530 |

### Capsule 1

| Component | mg/capsule |
|---|---|
| Compound X (MAO-B inhibitor) | 100 |
| Colloidal silicon dioxide | 1.5 |
| Lactose | 465.5 |
| Pregelatinized starch | 120.0 |
| Magnesium stearate | 3.0 |
| Total | 690 |

### Example 2: Enhanced Cognitive and Motor Rehabilitation in a Rat TBI Model

The ability of exemplary MAO-B inhibitors of the present invention to enhance rehabilitation after TBI is tested in the well characterized lateral fluid percussion model (LFP) (Mcintosh et al., Neuroscience, 1989, 28, 233-244; Hallam et al., J. Neurotrauma 2004, 21, 521-539).

Motor Rehabilitation

The staggered step (SS) assay is a skilled locomotor task that is useful to evaluate the ability of compounds to augment motor rehabilitation in the rat TBI model. (Klint et al., J. Neurotrauma 2003, 21st Annual National Neurotrauma Society Symposium, 10). It consists of a runway 8′ long and 3.5′ wide upon which a series of 28 raised steps are attached. Steps were alternately “staggered” 0.5 cm from midline and 25 cm between steps. Darkened home boxes (12″×12″×12″) were attached to both ends of the runway. A bright light and speaker with white noise generator were attached to the interior of the home box and exterior side of the home box such that it was enclosed within the runway. A computer controlled door was used to manage entrance/exit from the home boxes.

Rats are trained to a criterion performance on the staggered step (SS) task. After reaching criteria performance (“pre-injury baseline”), rats are injured using the LFP brain injury device and allowed to recover for 1 week. Seven days after injury (rehab day 1), all brain injured groups are shown to have significant impairments in performance of the SS task, compared to pre-injury baseline. The following day, rats are randomly assigned to receive either daily administration of vehicle/MAO-B inhibitors with rehabilitation or daily administration of vehicle/MAO-B inhibitors without rehabilitation.

Compared to vehicle alone, exemplary compounds of the present invention are able to enhance motor recovery in a dose dependent manner.

Cognitive Rehabilitation

The object recognition (OR) task is a non-spatial memory assay and can be used to assess whether MAO-B inhibitors (MAO-Bi) of the present invention can facilitate rehabilitation of cognitive function in brain-injured rats. LFP-injured mice have deficits in long-term memory—a measure of cognitive function. The object recognition task is used because it: 1) requires long term memory (LTM) formation, 2) allows for repeated training and testing of memory performance, and 3) ensures that performance on an individual trial is not confounded by memory performance on a previous trial. Object recognition is a non-aversive task that relies on a rat's natural exploratory behavior. (Antunes and Bial, Cogn. Process. 201, 13, 93-110.) During training for this task, rats are presented with two identical objects. Given adequate exposure (training time), normal rats form a LTM of an explored object. When rats are presented with two different objects (i.e., one novel object and one previously explored object) rats will choose to spend more time exploring a novel object. This task can be performed repeatedly on the same animals by exposing them serially to different sets of novel objects.

Prior to injury, rats are trained/tested for 5 trials for object recognition memory. A pre-injury baseline performance for each group is obtained. Upon completion of trial 5, rats are injured with the LFP device and allowed to recover for 7 days. On the first baseline trial following injury, both groups display similar long term memory deficits for object recognition.

Drug assisted cognitive rehabilitation is started with MAO-B inhibitors. Rats are given 5 trials of OR training/testing and are administered either vehicle or an MAO-B inhibitor prior to each training session.

Compared to vehicle-injected controls, the MAO-B inhibitor group shows significantly better cognitive performance during rehabilitation. Moreover, the MAO-B inhibitor group performs significantly better than the vehicle group when trained and tested after rehabilitation in the absence of drug—even weeks later.

### Example 3. Enhanced Motor Rehabilitation in a Rat Stroke Model

The efficacy of compounds of the present invention to enhance motor recovery after stroke is tested in a well-established rodent stroke model based on cortical ischemia and motor impairment. (e.g., Boychuk et al., Neurorehabil. Neural Repair. 2011, 25, 88-97; MacDonald et al., Neurorehabil. Neural Repair. 2007, 21, 486-496). The approach combines extensive behavioral and neurophysiological methods that allows for a comprehensive examination of the neural correlates mediating motor improvement post stroke.

To establish baseline levels of motor performance, rats are trained on one or more voluntary motor forelimb tasks, which can include the following:

1. Single Pellet Reaching: Prior to reach training and testing, animals are placed on a restricted diet (90% original body weight). Initially, a period of pre-training occurs in test cages where animals are trained until they successfully retrieve 10 pellets in one session from large trays of pellets (approximately 1 hour per day for 2-4 days). Immediately following pre-training, all animals are trained to criterion (40% accuracy over a three day period) on a single pellet reaching task (approximately 2 weeks). Each session is videotaped and subsequently used to assess reaching accuracy. A successful reach is scored when the animal grasps the food pellet, brings it into the cage and to its mouth without dropping the pellet. The primary outcome variable for this task is percentage reaching accuracy and is calculated as: [(# successful retrievals/the total # of reaches)×100]. The number of reach attempts is also recorded.

2. Cylinder Forepaw Placement Test: To test voluntary forelimb use, animals are placed into a transparent cylinder (20 cm×30 cm) for 5 minutes and video recorded for subsequent analysis of voluntary forelimb use during vertical exploration. The primary outcome variable for the cylinder test is the cylinder forepaw asymmetry ratio (CAR) as described, for example, in Plowman et al., Behav. Brain Res. 2013, 237, 157-163.

3. Sunflower Seed Opening: To test object manipulation abilities, animals are placed into a clear plastic arena with five sunflower seeds located in the upper right hand corner. The primary outcome measure for sunflower seed testing is total manipulation time (TMT) and is defined as the total time spent manipulating, opening and placing the seed into the mouth (starting the moment the animal touches a seed and ending the second the animal drops the shell and releases the seed into the mouth) and represents the cumulative time across all five trials. Animals are tested over two consecutive days and a two-day average TMT calculated for each time point.

4. Vermicelli Handling Task: Animals are presented with five 7-cm uncooked vermicelli strands in their home cage and video recorded for subsequent analyses. To acclimate them to pasta handling, animals are given five strands of vermicelli in their home cages for several days prior to testing. The primary outcome measure for the vermicelli handling test is vermicelli asymmetry ratio (VAR) as described by Allred et al., J. Neurosci. Methods 2008, 170, 229-244. The VAR (%) is defined as the [(number of dominant forelimb adjustments/total number of dominant and non-dominant forelimb adjustments)×100]. Time to eat (beginning when the pasta piece is grasped and ending when piece is released by the paws and disappears into the mouth in seconds) is a secondary outcome measure for this task. For data analysis, the mean across the five trials is used.

Following determination of baseline performance levels, the animals receive a cortical infarction via photothrombosis of the middle cerebral artery contralateral to the preferred paw (as determined during baseline training). The animals are then retested on one or more of the motor tasks for three days to establish a post-injury level of motor performance. Based on post injury performance on the single pellet reaching task, animals are assigned to a vehicle group (control) or to different drug dose groups in a manner that ensures an equivalent level of impairment across conditions.

Motor rehabilitation consists of thirty minutes of training on the single pellet reaching task (as described above) for 8 weeks. One hour prior to training, drug or vehicle is administered to experimental and control animals, respectively. Following eight weeks of rehabilitation, all animals are again tested on one or more of the motor tasks to assess post-treatment motor performance. Intracortical microstimulation (ICMS) can also be used to derive high resolution forelimb motor maps from the motor cortex of the ipsilesional and contralesional hemisphere. Lesion volume can be measured using standard lesion reconstruction methods and correlated with both measures of impairment and motor map area.

The efficacy of each compound is assessed by comparing the rate and level of motor recovery in drug treated and vehicle treated animals. Compared to vehicle alone, exemplary compounds of the present invention are able to enhance motor recovery in a dose dependent manner. Exemplary compounds also can induce an expansion of movement representation extending beyond residual cortex that is not observed in vehicle injected controls.

### Example 4. Enhanced Rehabilitation in Human Subjects Undergoing Motor Rehabilitation and Functional Rehabilitation in a Post-Acute Stroke Setting

A randomized, double-blind, placebo-controlled, 21-day dosing study uses fMRI techniques and standard stroke rehabilitation outcome measures to evaluate the effect of compositions of the present invention on motor recovery and behavior in medically stable subjects following ischemic stroke.

Enrolled subjects, who are able to meet inclusion criteria, have unilateral upper extremity motor impairment; begin drug treatment within 2-12 weeks post-stroke; and are in a standard upper extremity rehabilitation program under the supervision of licensed therapists at least two weeks prior to randomization.

Approximately equal numbers of post-stroke subjects are enrolled in two groups based on time since the ischemic event: Group 1, Subacute: between 2 and 6 weeks post-stroke; and Group 2, Chronic: between 6 and 12 weeks post-stroke. A group of matched healthy control subjects is also enrolled but does not undergo drug or placebo administration.

Subacute and chronic stroke subjects are randomized to receive either active ingredient or placebo capsules, to be taken once daily in the morning at approximately the same time each day for 21 consecutive days. Blood samples for PK-related evaluations are also collected from stroke subjects at specified times over the course of the study.

Prior to dosing, subjects are assessed for baseline performance in multiple functional tests, such as FMA-UE, AMAT-9, Stroke Impact Scale (SIS, hand domain), grip strength, index finger tapping, somatosensory evoked potential (SSEP), gait test (if applicable), and 9-hole peg test. The subjects also receive the first pre-dose fMRI scanning, consisting of activated motor skill paradigms of hand grip and finger movement techniques. Additional MRI sequences are obtained to measure brain injury and stroke penumbra for estimation of stroke volume and cortical spinal tract injury.

During the 21-day dosing period, subjects carry out their prescribed rehabilitation therapy (as supervised by licensed physical or occupational therapists) and also perform the functional tests, including the index finger-tapping test, the FMA-UE, the 9-hole peg board test, the gait test (if indicated), grip strength, and the AMAT-9. On days 14 and 21, the subjects also receive a second and third fMRI scan with the activated motor skill paradigm.

The efficacy of each compound is assessed by comparing the rate and level of motor recovery in drug treated and vehicle treated groups. Compared to vehicle alone, exemplary compounds of the present invention significantly enhance motor recovery in subacute and chronic stroke patients. Exemplary compounds also induce expanded fMRI movement representations beyond that observed with vehicle alone.

All publications, patent and patent applications mentioned in this specification are incorporated herein by reference to the same extent as if each individual publication, patent or patent application was specifically and individually incorporated by reference.

While this invention has been particularly shown and described with references to preferred embodiments thereof, it will be understood by those skilled in the art that various changes in form and details can be made therein without departing from the scope of the invention encompassed by the appended claims. Further, all embodiments included herein are given solely for the purpose of illustration and are not to be construed as limitations of the present invention, as many variations thereof are possible without departing from the spirit and scope of the invention.

1. (canceled)

2. A method of treating a neurological deficit associated with a neurotrauma disorder during rehabilitation, comprising:
(a) administering to a subject in need thereof an effective amount of an MAO-B inhibitor;
(b) training the subject under conditions sufficient to improve performance of a neurological function whose impairment is associated with the deficit; and
(c) repeating steps (a) and (b) one or more times,
whereby the amount of training sufficient to improve said performance is reduced compared to that produced by training alone.

3. (canceled)

4. The method of claim 2, wherein the subject is a post-acute trauma patient and/or the rehabilitation is post-acute trauma rehabilitation.

5.-6. (canceled)

7. The method of claim 2, wherein the neurotrama disorder is stroke.

8. The method of claim 7, wherein the stroke is hemorrhagic stroke.

9. The method of claim 7, wherein the stroke is ischemic stroke.

10. The method of claim 7, wherein the subject is a post-acute stroke patient, and/or the rehabilitation is post-acute stroke rehabilitation.

11. (canceled)

12. The method of claim 2, wherein said treating begins no earlier than about 2 days after onset of the neurotrauma disorder.

13.-14. (canceled)

15. The method of claim 12, wherein said treating begins no earlier than about 1 month after onset of the neurotrauma disorder.

16.-19. (canceled)

20. The method of claim 2, wherein the neurotrama disorder is TBI.

21. The method of claim 20, wherein the subject is a post-acute TBI patient, and/or the rehabilitation is post-acute TBI rehabilitation.

22. (canceled)

23. The method of claim 20, wherein the TBI is a penetrating injury.

24. (canceled)

25. The method of claim 20, wherein the TBI is a closed head injury.

26. The method of claim 20, wherein the TBI is a blast injury.

27.-37. (canceled)

38. A method of treating a neurological deficit during rehabilitation of stroke in a post-acute stroke setting, comprising: (a) administering to a subject in need thereof an effective amount of an MAO-B inhibitor; (b) training the subject under conditions sufficient to improve performance of a neurological function whose impairment is associated with the deficit; and (c) repeating steps (a) and (b) one or more times, whereby the amount of training sufficient to improve said performance is reduced compared to that produced by training alone.

39. (canceled)

40. A method of rehabilitating a patient suffering from a neurological deficit associated with a neurotrauma disorder, comprising administering to the patient an effective amount of an MAO-B inhibitor during rehabilitation, wherein the patient has been medically stabilized from the neurotrauma disorder prior to the first administration of the MAO-B inhibitor.

41. The method of claim 40, wherein the neurotrauma disorder is a stroke, and the patient is administered the MAO-B inhibitor during post-acute stroke rehabilitation.

42. The method of claim 40, wherein the neurotrauma disorder is a traumatic brain injury (TBI), and the patient is administered the MAO-B inhibitor during post-acute TBI rehabilitation.

43.-47. (canceled)

48. The method of claim 2, wherein the neurological deficit is a motor deficit.

49. The method of claim 2, wherein the neurological deficit is a cognitive deficit.

50. The method of claim 2, wherein the cognitive deficit is a deficit in memory formation.

51. The method of claim 2, wherein the deficit in memory formation is a deficit in long-term memory formation.

52. The method of claim 2, wherein the MAO-B inhibitor is administered chronically.

53. The method of claim 2, wherein rehabilitation includes one or more of physical therapy, occupational therapy and cognitive therapy.

54.-58. (canceled)

59. The method of claim 2, wherein the subject is a human.

60. The method of claim 2, wherein the MAO-B inhibitor is a reversible inhibitor.

61. The method of claim 2, wherein the MAO-B inhibitor has greater than 10-fold selectivity for MAO-B over MAO-A.

62.-109. (canceled)

110. The method of claim 38, wherein the neurological deficit is a motor deficit.

111. The method of claim 38, wherein the neurological deficit is a cognitive deficit.

112. The method of claim 38, wherein the cognitive deficit is a deficit in memory formation.

113. The method of claim 38, wherein the deficit in memory formation is a deficit in long-term memory formation.

114. The method of claim 38, wherein the MAO-B inhibitor is administered chronically.

115. The method of claim 38, wherein rehabilitation includes one or more of physical therapy, occupational therapy and cognitive therapy.

116. The method of claim 40, wherein the MAO-B inhibitor is administered chronically.
